(19) United States
(12) Patent Application Publication
(10) Pub. No.: US 2004/0062778 A1
(43) Pub. Date: Apr. 1, 2004

(54) Surface dissolution and/or bulk erosion controlled release compositions and devices

(76) Inventors: Adi Shefer, East Brunswick, NJ (US); Samuel David Shefer, East Brunswick, NJ (US)

Correspondence Address: Diane Dunn McKay, Esq., Mathews, Collins, Shepherd & McKay, P.A., 100 Thanet Circle, Suite 306, Princeton, NJ, 08540, US

(21) Appl. No.: 10/255,289

(22) Filed: Sep. 26, 2002

(51) Int. Cl. A61K 9/00; A61K 9/127

(52) U.S. Cl. 424/400

(57) ABSTRACT

The present invention relates to a controlled release system comprising matrix compositions which control the lag time and release rate of the composition, as well as pharmaceutical and other active ingredients included in the composition, through surface dissolution and/or bulk erosion of the system. The controlled release system can be used to target and control the release of active ingredients onto certain regions of the gastrointestinal tract including the stomach and the small intestine. The matrix compositions of the present invention can be comprised of the following components: a wax material, fat material, water sensitive material and surface active material.

### BACKGROUND OF THE INVENTION

1. Field of the Invention

The present invention relates to a controlled release system including a matrix composition for controlling the lag time and release rate of active ingredients encapsulated in the composition through surface dissolution and/or bulk erosion of the system. Articles can be formed of the composition. The controlled release compositions and articles of the present invention are useful for pharmaceutical, diagnostic, food, nutraceuticals, and other consumer and diversified product applications.

2. Description of the Related Art

Extensive work has been directed in recent years towards controlling the time lag and release rate of active ingredients mainly through coating technology, especially using pH sensitive polymers. These coatings have been modified to achieve longer time lags prior to release so that the beneficial agent can be released in the lower end of the small intestine or in the colon. However, these coatings were observed to function similarly as common enteric coatings.

Typically pH-sensitive materials have been used as coatings to protect beneficial agents, or to encapsulate irritating beneficial agents during transit through the stomach, and then release the agent shortly after entering the small intestine. pH-sensitive coatings that achieve delivery in the colon have been described in patents such as U.S. Pat. No. 4,910,021 and WO 9001329. U.S. Pat. No. 4,910,021 describes using a pH-sensitive material to coat a capsule. WO 9001329 describes using pH-sensitive coatings on beads containing acid, where the acid in the bead core prolongs dissolution of the pH-sensitive coating.

U.S. Pat. No. 6,068,859 discloses a controlled-release dosage form of azithromycin having an improved side effect profile; a process for preparing the dosage form; and a method of treating a microbial infection, comprising administering azithromycin in such a controlled-release dosage form to a mammal, including a human patient, in need of such treatment. A first delayed release embodiment according to the invention is a pH-dependent coated tablet which comprises a tablet core comprising azithromycin, a disintegrant, a lubricant, and one or more pharmaceutical carriers, such core being coated with a material, preferably a polymer, which is substantially insoluble and impermeable at the pH of the stomach, and which is more soluble and permeable at the pH of the small intestine. Preferably, the coating polymer is substantially insoluble and impermeable at pH<5.0, and water-soluble at pH>5.0.

U.S. Pat. No. 5,175,003 discloses a dual mechanism polymer mixture composed of pH-sensitive enteric materials and film-forming plasticizers capable of conferring permeability to the enteric material, for use in drug-delivery systems; a matrix pellet composed of a dual mechanism polymer mixture permeated with a drug and sometimes covering a pharmaceutically neutral nucleus; a membrane-coated pellet comprising a matrix pellet coated with a dual mechanism polymer mixture envelope of the same or different composition; and a pharmaceutical dosage form containing matrix pellets. The matrix pellet releases acid-soluble drugs by diffusion in acid pH and by disintegration at pH levels of nominally about 5.0 or higher.

U.S. Pat. No. 4,503,030 discloses an osmotic device for dispensing a drug to certain pH regions of the gastrointestinal tract. More particularly, the invention relates to an osmotic device comprising a wall formed of a semi-permeable pH sensitive composition that surrounds a compartment containing a drug, with a passageway through the wall connecting the exterior of the device with the compartment. The device delivers the drug at a controlled rate in the region of the gastrointestinal tract having a pH of less than 3.5, and the device self-destructs and releases all its drug in the region of the gastrointestinal tract having a pH greater than 3.5, thereby providing total availability for drug absorption.

U.S. Pat. Nos. 5,609,590 and 5,358,502 disclose an osmotic bursting device for dispensing a beneficial agent to an aqueous environment. The device comprises a beneficial agent and osmagent surrounded at least in part by a semi-permeable membrane. Alternatively the beneficial agent may also function as the osmagent. The semi-permeable membrane is permeable to water and substantially impermeable to the beneficial agent and osmagent. A trigger means is attached to the semi-permeable membrane (e.g., joins two capsule halves). The trigger means is activated by a pH of from 3 to 9 and triggers the eventual, but sudden, delivery of the beneficial agent. These devices enable the pH-triggered release of the beneficial agent core as a bolus by osmotic bursting.

U.S. Pat. No. 5,316,774 discloses a composition for the controlled release of an active substance comprising a polymeric particle matrix, where each particle defines a network of internal pores. The active substance is entrapped within the pore network together with a blocking agent having physical and chemical characteristics selected to modify the release rate of the active substance from the internal pore network. In an exemplary embodiment, drugs may be selectively delivered to the intestines using an enteric material as the blocking agent. The enteric material remains intact in the stomach but will degrade under the pH conditions of the intestines. In another exemplary embodiment, the sustained release formulation employs a blocking agent, which remains stable under the expected conditions of the environment to which the active substance is to be released. The use of pH-sensitive materials alone to achieve site-specific delivery is difficult because of leaking of the beneficial agent prior to the release site or desired delivery time and it is difficult to achieve long time lags before release of the active ingredient after exposure to high pH (because of rapid dissolution or degradation of the pH-sensitive materials).

There are also hybrid systems which combine pH-sensitive materials and osmotic delivery systems. These devices provide delayed initiation of sustained-release of the beneficial agent. In one device a pH-sensitive matrix or coating dissolves releasing osmotic devices that provide sustained release of the beneficial agent (U.S. Pat. Nos. 4,578,075, 4,681,583, and 4,851,231). A second device consists of a semipermeable coating made of a polymer blend of an insoluble and a pH-sensitive material. As the pH increases, the permeability of the coating increases, increasing the rate of release of beneficial agent (U.S. Pat. Nos. 4,096,238, 4,503,030, 4,522,625, and 4,587,117).

U.S. Pat. No. 5,484,610 discloses terpolymers which are sensitive to pH and temperature which are useful carriers for conducting bioactive agents through the gastric juices of the stomach in a protected form. The terpolymers swell at the higher physiologic pH of the intestinal tract causing release of the bioactive agents into the intestine. The terpolymers are linear and are made up of 35 to 99 wt % of a temperature sensitive component, which imparts to the terpolymer LCST (lower critical solution temperature) properties below body temperatures, 1 to 30 wt % of a pH sensitive component having a pKₐ in the range of from 2 to 8 which functions through ionization or deionization of carboxylic acid groups to prevent the bioactive agent from being lost at low pH but allows bioactive agent release at physiological pH of about 7.4 and a hydrophobic component which stabilizes the LCST below body temperatures and compensates for bioactive agent effects on the terpolymers. The terpolymers provide for safe bioactive agent loading, a simple procedure for dosage form fabrication and the terpolymer functions as a protective carrier in the acidic environment of the stomach and also protects the bioactive agents from digestive enzymes until the bioactive agent is released in the intestinal tract.

U.S. Pat. No. 6,103,865 discloses pH-sensitive polymers containing sulfonamide groups, which can be changed in physical properties, such as swellability and solubility, depending on pH and which can be applied for a drug-delivery system, bio-material, sensor, etc, and a preparation method therefore. The pH-sensitive polymers are prepared by introduction of sulfonamide groups, various in pKa, to hydrophilic groups of polymers either through coupling to the hydrophilic groups, such as acrylamide, N,N-dimethylacrylamide, acrylic acid, N-isopropylacrylamide, etc, of polymers or copolymerization with other polymerizable monomers. These pH-sensitive polymers may have a structure of linear polymer, grafted copolymer, hydrogel or interpenetrating network polymer.

U.S. Pat. No. 5,656,292 discloses a composition for pH dependent or pH regulated controlled release of active ingredients especially drugs. The composition consists of a compactable mixture of the active ingredient and starch molecules substituted with acetate and dicarboxylate residues. The preferred dicarboxylate acid is succinate. The average substitution degree of the acetate residue is at least 1 and 0.2-1.2 for the dicarboxylate residue. The starch molecules can have the acetate and dicarboxylate residues attached to the same starch molecule backbone or attached to separate starch molecule backbones. The present invention also discloses methods for preparing said starch acetate dicarboxylates by transesterification or mixing of starch acetates and starch dicarboxylates respectively.

U.S. Pat. Nos. 5,554,147, 5,788,687, and 6,306,422 disclose a method for the controlled release of a biologically active agent wherein the agent is released from a hydrophobic, pH-sensitive polymer matrix. The polymer matrix swells when the environment reaches pH 8.5, releasing the active agent. A polymer of hydrophobic and weakly acidic comonomers is disclosed for use in the controlled release system. Also disclosed is a specific embodiment in which the controlled release system may be used. The pH-sensitive polymer is coated onto a latex catheter used in ureteral catheterization. A ureteral catheter coated with a pH-sensitive polymer having an antibiotic or urease inhibitor trapped within its matrix will release the active agent when exposed to the high pH urine as the polymer gel

Conventional surface and/or bulk erosion based controlled release systems have been described which are composed of bioerodible polymers such as: polyamidespoly(lactide-co-glycolide) (PLGA), polyanhydrides, polyesters, and polyorthoesters. Polymers recognized as eroding by bulk erosion include polylactic acid, polyglutamic acid, polycaprolactone and lactic/glycolic acid copolymers, see Pitt et al, Biomaterials 2:215-220 (1981); Koenig et al, J. Macromol. Sci. Phys. 2:391-407 (1966). Polymers recognized as eroding by surface erosion are polyorthoesters. An example of such polyorthoesters having a carbonyloxy functionality is described in U.S. Pat. No. 4,070,347. As has been recognized, their advantages lie in that not only are they hydrophobic, but also that hydrolysis of orthoester is pH sensitive, a property which has been proven useful in regulating the release of active substance.

U.S. Pat. No. 4,891,225 discloses a hydrophobic polymeric matrix which is suitable for use after implantation in vivo in a subject for the controlled release and delivery of biologically active substances such as drugs, antibiotics, steroids and the like. Alternatively, the matrix can be used outside the body for release of fragrances, pesticides and the like. The implantable matrix comprises a polymeric polyanhydride formulation whose internal anhydride linkages are hydrolytic in nature in varying degrees in accordance with the chemical composition of the backbone, pH and temperature of the environment. As the individual anhydride linkages become hydrolyzed, the matrix erodes predominantly by surface erosion into non-toxic biocompatible degradation products with concomitant release of the biologically active substance.

Typical biologically degradable polymers include homopolyesters and copolyesters, in particular of lactic acid and glycolic acid, as are described in U.S. Pat. Nos. 3,773,919 and 3,297,033 respectively. A disadvantage in these polymers is the low or poorly controllable swellability of the polyesters in the physiological environment, which hinders permeation of the active compounds incorporated in the implant and causes an only low liberation rate after the initial “burst effect”. Polyacetals and polyketals have been described in U.S. Pat. No. 4,304,767. Polyanhydrides have been described by H. G. Rosen et al., Biomaterials 4, 131 (1983), and polyorthoesters have been described in U.S. Pat. No. 4,180,646; all these compounds were developed as biologically degradable polymers for use as implant materials. Due to the lack of further functional groups, similar to the polyesters mentioned, the degradation of these polymers is only determined by the hydrolytic resistance of the carbonyl function in the main polymer chain. In addition, such polymers do not have adequate stability for implantation periods of months. Other classes of polymers, polyamides, in particular polyamino acids, have been described in U.S. Pat. No. 3,371,069 as bioresorbable implant materials. However, the industrial preparation of polyamino acids requires the use of expensive protected amino acids, relatively large amounts of highly toxic phosgene, the removal of the protecting groups and the chemical modification of the polymers obtained.

The prior art of which applicant is aware does not set forth matrix compositions and articles for controlling the lag time and release rate of pharmaceutical and other active ingredients though surface dissolution and/or bulk erosion of the system or the device, to target and control the release of active ingredients onto certain regions of the gastrointestinal tract including the stomach and the small intestine. Therefore, there remains a need for a matrix system for the controlled release of active ingredients where the release occurs by surface and/or bulk erosion, and the composition erodes into products that are non-toxic and readily eliminated by the body in vivo.

### SUMMARY OF THE INVENTION

The present invention relates to a controlled release system comprising matrix compositions which control the lag time and release rate of the composition, as well as pharmaceutical, other active ingredients or devices included in the composition, through surface dissolution and/or bulk erosion of the system. The controlled release system can be used to target and control the release of active ingredients or devices onto certain regions of the gastrointestinal tract including the stomach and the small intestine.

The matrix compositions of the present invention can be comprised of the following components: a wax material, fat material, water sensitive material and surface active material. The wax material can have a melting point between about 25 to about 150 degrees C. and a penetration point of about 1 to about 10. The wax material controls the erosion rate and the mechanical properties and physical integrity of the matrix composition. The fat material controls the water solubility of the matrix composition. The water sensitive material and surface active material are used to control water solubility. The matrix composition can be used to deliver active ingredients that are soluble or dispersed in the composition or to deliver encapsulated active ingredients in the form of microspheres or nanospheres which are soluble or dispersed in the composition. The matrix composition can be formed into an article or used as a carrier for a device. Devices which can be used in the present invention include implant devices, imaging devices, and medical devices.

The matrix compositions and articles formed of the matrix composition disintegrate into small pieces or particles after a predetermined time and release a material or device associated with the matrix composition. The release rate of active ingredients as well as the time lag can be accurately controlled through fine-tuning of the matrix composition. The release rate can also be controlled by the shape and surface area of a system or an article formed of the matrix composition.

The composition of the present invention can be molded into any shape or form which has the advantages of: (i) protection of the active ingredients, during storage, or until needed and the composition reaches the target site; (ii) controlled lag time, and (iii) controlled release of active ingredients, over an extended period of time.

The invention also provides a method for producing the controlled release compositions of the present invention that comprises the steps of:

(i) heating a wax material to a temperature above the melting point of the materials to form a melt;

(ii) heating a fat material to a temperature above the melting point of the materials to form a melt;

(iii) dissolving or dispersing a surface active agent into the melt of the wax or fat;

(iv) combining the melt of the wax with the melt of the fat;

(v) dissolving or dispersing one or more first active agents into the combined melt;

(vi) optionally dissolving or dispersing one or more second encapsulated active agents into the melt of the wax or fat;

(vii) dispersing a water sensitive material into the combined melt of the wax or fat; and

(viii) cooling the melt to form a dry powder composition.

A method for producing an article formed of the matrix composition comprises the steps of:

(i) heating the wax material to a temperature above the melting point of the materials to form a melt;

(ii) heating the fat material to a temperature above the melting point of the materials to form a melt;

(iii) dissolving or dispersing a surface active agent into the melt of the wax or fat;

(iv) combining the melt of the wax with the melt of the fat;

(v) dissolving or dispersing one or more first active agents into the combined melt;

(vi) optionally dissolving or dispersing one or more second encapsulated active agents into the melt of the wax or fat;

(vii) dispersing a water sensitive material into the combined melt of the wax or fat;

(viii) cooling the melt to form a dry powder composition; or

(ix) molding the melt into a predetermined geometric dimensions or shapes.

The release system of the present invention does not cause adverse tissue reactions within the body in vivo, exhibits mechanical and physical integrity of the matrix, and releases the active substance with a controllable kinetic mechanism. In addition, the delivery system can be easily processed into an article having predetermined geometric dimensions. The articles formed of the matrix composition remain stable upon storage under a variety of conditions.

The present invention addresses the ongoing need for matrix compositions and articles for controlling the lag time and release rate of pharmaceutical and other active ingredients or devices though surface dissolution and/or bulk erosion of the system or the article, to target and control the release of active ingredients onto certain regions of the gastrointestinal tract including the stomach and the small intestine. The invention also provides pharmaceutical, diagnostic, imaging, and other consumer and diversified products comprising the controlled release compositions and articles of the present invention.

### DETAILED DESCRIPTION

The present invention relates to matrix compositions and articles formed of the matrix compositions for controlling the lag time and release rate of active ingredients, such as pharmaceuticals, in the composition through surface dissolution and/or bulk erosion of the system or article. The matrix composition can be used to target and control the release of active ingredients or devices onto certain regions of the gastrointestinal tract including the stomach and the small intestine.

The matrix compositions of the present invention can be comprised of the following components: a wax material, fat material, water sensitive material and optionally a surface active material or a device. The wax material controls the erosion rate as well as the mechanical properties and physical integrity of the matrix composition. The fat controls the water solubility of the matrix composition. The water sensitive material and surface active material are used to control water solubility. By altering the proportion of a water sensitive material to the wax material the time lag subsequent to triggering and prior to erosion can be tailored. The composition can be utilized as carrier for a device or to deliver active ingredients that are soluble or dispersed in the composition and/or encapsulated active ingredients which are in the form of microspheres or nanospheres. For example, the device can be an implant device, imaging device or medical device.

The matrix compositions of the present invention can comprises from about 1% to about 50% by weight of wax, from about 1% to about 50% of fat, from about 1% to about 50% by weight water sensitive matrix, from about 0% to about 50% by weight active ingredients, and from about 0% to about 30% by weight surface active agents. The composition can include from about 1% to about 50% by weight active ingredients.

The matrix compositions of the present invention can be easily processed into articles having predetermined geometric dimensions and remain stable upon storage under a variety of conditions. The matrix compositions disintegrate with a controllable kinetic mechanism by surface and/or bulk erosion into small pieces or particles that are non-toxic and readily eliminated by the body in vivo.

The characteristics of the matrix compositions of the present invention, i.e., time lag, release mechanism (surface erosion or bulk erosion), and release rate are dependent in part on the characteristic of individual materials of the composition, in terms of water solubility, melting point, glass transition temperatures, mechanical properties, and ratio between the wax to fats and water sensitive materials. The surface area and shape of the system can determine the release rate for an article formed of the matrix composition.

The compositions and devices of the present invention can be further coated with a pH sensitive material to release the composition or device when the pH of the surrounding environment reaches a desired level.

In an aqueous environment the water sensitive matrix compositions dissolve or swell. The dissolution or swelling of the matrix disrupts the matrix structure and facilitates the disintegration of the compositions or the devices and release of the active ingredients or the encapsulated active ingredients.

The invention also provides pharmaceutical, diagnostic, implants, imaging, and other consumer and diversified products comprising the controlled release compositions and articles of the present invention.

Wax Materials

Considerations in the selection of the matrix material include good barrier properties to the active ingredients, low toxicity and irritancy, stability, integrity, and high loading capacity for the active agents of interest. Suitable wax materials for the compositions and devices of the present invention are inert nontoxic materials with a melting point range between about 25 degrees C. and about 150 degrees C. and penetration point of about 1 to about 10. Examples of wax materials include natural waxes, synthetic waxes and mixtures thereof. Suitable waxes also include natural, regenerated, or synthetic food approved waxes including animal waxes such as beeswax, vegetable waxes such as carnauba, candelilla, sugar cane, rice bran, and bayberry wax, mineral waxes such as petroleum waxes including paraffin and microcrystalline wax, and mixtures thereof.

Other wax materials that are known to those skilled in the art and suitable materials as described in “Industrial Waxes” Vol. I and II, by Bennett F.A.I.C., published by Chemical Publishing Company Inc., 1975 and Martindale, “The Extra Pharmacopoeia”, The Pharmaceutical Press, 28^{th} Edition pp. 1063-1072, 1982 can be used in the present invention.

Fat Materials

Suitable fat materials and/or glyceride materials which can be used in the present invention include, but are not limited to, the following classes of lipids: mono-, di and triglycerides, phospholipids, sphingolipids, cholesterol and steroid derivatives, terpenes and vitamins.

The fat material of the present invention can be a glyceride selected from monoglycerides, diglycerides, glyceryl monostearate, glyceryl tristearate and mixtures thereof. Other fat materials which can be used are hydrogenated palm oil, hydrogenated palm kernel oil, hydrogenated peanut oil, hydrogenated rapeseed oil, hydrogenated rice bran oil, hydrogenated soybean oil, hydrogenated cottonseed oil, hydrogenated sunflower oil, partially hydrogenated soybean oil, partially hydrogenated cottonseed oil, and mixtures thereof.

Examples of solid fat materials which can be used in the present invention, include solid hydrogenated castor and vegetable oils, hard fats, and mixtures thereof. Other fat materials which can be used, include triglycerides of food grade purity, which can be produced by synthesis or by isolation from natural sources. Natural sources can include animal fat or vegetable oil, such as soy oil, as a source of long chain triglycerides (LCT). Other triglycerides suitable for use in the present invention are composed of a majority of medium length fatty acids (C10-C18), denoted medium chain triglycerides (MCT). The fatty acid moieties of such triglycerides can be unsaturated or polyunsaturated and mixtures of triglycerides having various fatty acid material.

Phospholipids which may be used include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and beta-acyl-y-alkyl phospholipids. Examples of phospholipids include, but are not limited to, phosphatidylcholines such as dioleoylphosphatidylcholine, dimyristoylphosphatidylcholine, dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophosphoethanolamine. Synthetic phospholipids with asymmetric acyl chains (e.g., with one acyl chain of 6 carbons and another acyl chain of 12 carbons) can also be used.

Steroids which can be used include as fat materials, but are not limited to, cholesterol, cholesterol sulfate, cholesterol hemisuccinate, 6-(5-cholesterol 3 beta-yloxy) hexyl6-amino-6-deoxy-1-thio-alpha-D-galactopyranoside, 6-(5-cholesten-3 beta-tloxy)hexyl-6-amino-6-deoxyl-1-thio-alpha-D mannopyranoside and cholesteryl)4′-trimethyl 35 ammonio)butanoate.

Additional lipid compounds as fat material which can be used include tocopherol and derivatives, and oils and derivatized oils such as stearlyamine.

The fat material can be fatty acids and derivatives thereof which can include, but are not limited to, saturated and unsaturated fatty acids, odd and even number fatty acids, cis and trans isomers, and fatty acid derivatives including alcohols, esters, anhydrides, hydroxy fatty acids and prostaglandins. Saturated and unsaturated fatty acids that can be used include, but are not limited to, molecules that have between 12 carbon atoms and 22 carbon atoms in either linear or branched form. Examples of saturated fatty acids that can be used include, but are not limited to, lauric, myristic, palmitic, and stearic acids. Examples of unsaturated fatty acids that can be used include, but are not limited to, lauric, physeteric, myristoleic, palmitoleic, petroselinic, and oleic acids. Examples of branched fatty acids that can be used include, but are not limited to, isolauric, isomyristic, isopalmitic, and isostearic acids and isoprenoids. Fatty acid derivatives include 12-(((7′-diethylaminocoumarin-3yl)carbonyl)methylamino)-octadecanoic acid; N-[12-(((7′diethylaminocoumarin-3-yl)carbonyl)methyl-amino)octadecanoyl]-2-aminopalmitic acid, N succinyl-dioleoylphosphatidylethanol amine and palmitoyl-homocysteine; and/or combinations thereof. Mono, di and triglycerides or derivatives thereof that can be used include, but are not limited to, molecules that have fatty acids or mixtures of fatty acids between 6 and 24 carbon atoms, digalactosyldiglyceride, 1,2-dioleoyl-sn-glycerol; 1,2-cdipalmitoyl-sn-3 succinylglycerol; and 1,3-dipalmitoyl-2-succinylglycerol.

Water Sensitive Materials

Water-sensitive materials for use in the present invention comprises of water soluble and water dispersible natural oligomers, synthetic oligomers, natural polymers, synthetic polymers and copolymers, starch derivatives, polysaccharides, hydrocolloids, natural gums, proteins, and mixtures thereof.

Suitable water soluble materials include xylose, ribose, glucose, mannose, galactose, fructose, dextrose, polydextrose, sucrose, maltose, or corn syrup solids, palatin, sorbitol, xylitol, mannitol, maltitol, lactitol, xanthan, maltodextrin, galactomanan or tragacanth, manitol, lactitol, and mixtures thereof. Water sensitive materials include oligisaccharides and hydrocolloids. Suitable oligosaccharides and hydrocolloids are xanthan, maltodextrin, galactomanan or tragacanth, manitol, lactitol, preferably maltodextrins such as Maltrin™ M100, Maltrin™ M150, and Maltrin™ M180, commercially available from the Grain Processing Corporation of Muscatine, Iowa, and Lactitol commercially available from the Purac Corporation and Cultor Food Science of Ardsley, N.Y. Suitable water soluble polymers include starch, starch derivatives, cellulose, cellulose derivatives.

Examples of synthetic water sensitive polymers which are useful for the invention include polyvinyl pyrrolidone, water soluble celluloses, polyvinyl alcohol, ethylene maleic anhydride copolymer, methylvinyl ether maleic anhydride copolymer, acrylic acid copolymers, anionic polymers of methacrylic acid and methacrylate, cationic polymers with dimethyl-aminoethyl ammonium functional groups, polyethylene oxides, water soluble polyamide or polyester.

Examples of water soluble hydroxyalkyl and carboxyalkyl celluloses include hydroxyethyl and carboxymethyl cellulose, hydroxyethyl and carboxyethyl cellulose, hydroxymethyl and carboxymethyl cellulose, hydroxypropyl carboxymethyl cellulose, hydroxypropyl methyl carboxyethyl cellulose, hydroxypropyl carboxypropyl cellulose, hydroxybutyl carboxymethyl cellulose, and the like. Also useful are alkali metal salts of these carboxyalkyl celluloses, particularly and preferably the sodium and potassium derivatives.

The polyvinyl alcohol useful in the practice of the invention is partially and fully hydrolyzed polyvinyl acetate, termed “polyvinyl alcohol” with polyvinyl acetate as hydrolyzed to an extent, also termed degree of hydrolysis, of from about 75% up to about 99%. Such materials are prepared by means of any of Examples I-XIV of U.S. Pat. No. 5,051,222 issued on Sep. 24, 1991, the specification for which is incorporated by reference herein.

Polyvinyl alcohol useful for practice of the present invention is Mowiol® 3-83, having a molecular weight of about 14,000 Da and degree of hydrolysis of about 83%, Mowiol® 3-98 and a fully hydrolyzed (98%) polyvinyl alcohol having a molecular weight of 16,000 Da commercially available from Gehring-Montgomery, Inc. of Warminister Pa. Other suitable polyvinyl alcohols are: AIRVOL® 205, having a molecular weight of about 15,000-27,000 Da and degree of hydrolysis of about 88%, and VINEX® 1025, having molecular weight of 15,000-27,000 Da degree of hydrolysis of about 99% and commercially available from Air Products & Chemicals, Inc. of Allentown, Pa.; ELVANOL® 51-05, having a molecular weight of about 22,000-26,000 Da and degree of hydrolysis of about 89% and commercially available from the Du Pont Company, Polymer Products Department, Wilmington, Del.; ALCOTEX® 78 having a degree of hydrolysis of about 76% to about 79%, ALCOTEX® F88/4 having a degree of hydrolysis of about 86% to about 88% and commercially available from the Harlow Chemical Co. Ltd. of Templefields, Harlow, Essex, England CM20 2BH; and GOHSENOL® GL-03 and GOHSENOL® KA-20 commercially available from Nippon Gohsei K.K., The Nippon Synthetic Chemical Industry Co., Ltd., of No. 9-6, Nozaki Cho, Kita-Ku, Osaka, 530 Japan.

Suitable polysaccharides are polysaccharides of the non-sweet, coloidally-soluble types, such as natural gums, for example, gum arabic, starch derivatives, dextrinized and hydrolyzed starches, and the like. A suitable polysaccharide is a water dispersible, modified starch commercially available as Capule®, N-Lok®, Hi-Cap™ 100 or Hi-Cap™ 200 commercially available from the National Starch and Chemical Company of Bridgewater, N.J.; Pure-Cote™, commercially available from the Grain Processing Corporation of Muscatine, Iowa. In the preferred embodiment the natural gum is a gum arabic, commercially available from TIC Gums Inc. Belcamp, Midland.

Surface Active Agent

Surfactants which can be used in the present invention as a solubility augmenting agent generally include all pharmaceutically-acceptable surfactants, in which the surfactant has an HLB value of at least 10, and preferably at least about 15. Discussions of HLB numbers and how they are determined for specific surfactants can be found in, for example, the publication of ICI Surfactants entitled The HLB System and, in particular, in Chapter 7 of that publication entitled “How to Determine HLB of an Emulsifier” (ICI Americas, Inc., Wilmington, Del., 1992).

In certain embodiments, the HLB value of the surfactant is from about 15 to 50, and in other embodiments the HLB value is from about 15.6 to about 40. Suitable pharmaceutically-acceptable anionic surfactants include, for example, those containing carboxylate, sulfonate, and sulfate ions. Those containing carboxylate ions are sometimes referred to as soaps and are generally prepared by saponification of natural fatty acid glycerides in alkaline solutions. Cations associated with these surfactants include sodium, potassium, ammonium and triethanolamine. The chain length of the fatty acids range from 12 to 18. Although a large number of alkyl sulfates are available as surfactants, a preferred surfactant is sodium lauryl sulfate, which has an HLB value of about 40.

Sodium lauryl sulfate is a water-soluble salt, produced as a white or cream powder, crystals, or flakes. Also known as dodecyl sodium sulfate, sodium lauryl sulfate can be a mixture of sodium alkyl sulfates consisting chiefly of sodium lauryl sulfate. Sodium lauryl sulfate is also known as sulfuric acid monododecyl ester sodium salt. Furthermore, sodium lauryl sulfate is readily available from commercial sources such as Sigma or Aldrich in both solid form and as a solution. The solubility of sodium lauryl sulfate is about 1 gm per 10 ml/water. The fatty acids of coconut oil, consisting chiefly of lauric acid, are catalytically hydrogenated to form the corresponding alcohols. The alcohols are then esterified with sulfuric acid (sulfated) and the resulting mixture of alkyl bisulfates (alkyl sulfuric acids) is converted into sodium salts by reacting with alkali under controlled conditions of pH.

Alternative anionic surfactants for use as surface active agents in the present invention include docusate salts such as the sodium salt thereof. Other suitable anionic surfactants include, without limitation, alkyl carboxylates, acyl lactylates, alkyl ether carboxylates, N-acyl sarcosinates, polyvalent alkyl carbonates, N-acyl glutamates, fatty acid, polypeptide condensates and sulfuric acid esters.

In other aspects of the invention amphoteric (amphipathic/amphiphilic surfactants), non-ionic surfactants and/or cationic surfactants can be used as the surface active agent in the coprocessed compositions of the present invention. Suitable pharmaceutically-acceptable non-ionic surfactants include, for example, polyoxyethylene compounds, lecithin, ethoxylated alcohols, ethoxylated esters, ethoxylated amides, polyoxypropylene compounds, propoxylated alcohols, ethoxylated/propoxylated block polymers, propoxylated esters, alkanolamides, amine oxides, fatty acid esters of polyhydric alcohols, ethylene glycol esters, diethylene glycol esters, propylene glycol esters, glycerol esters, polyglycerol fatty acid esters, SPAN's (e.g., sorbitan esters), TWEEN's (i.e., sucrose esters), glucose (dextrose) esters and simethicone. The HLB for one acceptable non-ionic surfactant, polysorbate 40, is about 15.6.

Other suitable pharmaceutically-acceptable surfactants include acacia, benzalkonium chloride, cholesterol, emulsifying wax, glycerol monostearate, lanolin alcohols, lecithin, poloxamer, polyoxyethylene, and castor oil derivatives.

pH and Salt Sensitive Materials for Coating the Composition and Devices

Any material and structural form can be used as a pH-sensitive material to coat the compositions and articles formed of the composition to maintain the integrity of these systems until triggered by a solution of the desired pH. Typically, the trigger pH is between about 3 to about 12, although in some applications it may be higher or lower. The trigger pH is the threshold pH value or range of values at which either above or below the trigger pH the pH-sensitive material degrades, and/or dissolves. The compositions and articles formed of the composition can be formed to be stable in solutions and then as the pH rises above the trigger pH the compositions and articles are activated. Once activated, the compositions are released.

In one embodiment, a pH-sensitive trigger means is used such that the compositions and articles are capable of becoming more permeable to water and/or losing physical strength following triggering by a solution of the desired pH, either above or below the trigger pH.

The pH-sensitive materials can be insoluble solids in acidic or basic aqueous solutions, which dissolve, or degrade and dissolve, as the pH of the solution is neutral. The pH-sensitive materials can be insoluble solids in acidic or basic aqueous solutions which dissolve, or degrade and dissolve, as the pH of the solution rises above or drops below a trigger pH value.

Exemplary pH-sensitive materials include copolymers of acrylate polymers with amino substituents, acrylic acid esters, polyacrylamides, phthalate derivatives (i.e., compounds with covalently attached phthalate moleties) such as acid phthalates of carbohydrates, amylose acetate phthalate, cellulose acetate phthalate, other cellulose ester phthalates, cellulose ether phthalates, hydroxy propyl cellulose phthalate, hydroxypropyl ethylcellulose phthalate, hydroxypropyl methyl cellulose phthalate, methyl cellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinyl acetate phthalate copolymer, styrene and maleic acid copolymers, formalized gelatin, gluten, shellac, salol, keratin, keratin sandarac-tolu, ammoniated shellac, benzophenyl salicylate, cellulose acetate trimellitate, cellulose acetate blended with shellac, hydroxypropylmethyl cellulose acetate succinate, oxidized cellulose, polyacrylic acid derivatives such as acrylic acid and acrylic ester copolymers, methacrylic acid and esters thereof, vinyl acetate and crotonic acid copolymers.

Examples of suitable pH sensitive polymers for use in the present invention are the Eudragit® polymers series from Rohm America Inc., a wholly-owned subsidiary of Degussa-Huls Corporation, headquartered in Piscataway, N.J., and an affiliate of Rohm GmbH of Darmstadt, Germany. EUDRAGIT® L 30 D-55 and EUDRAGIT® L 100-55, pH dependent anionic polymer that is soluble at pH above 5.5 and insoluble blow pH 5. These polymers can be utilized for targeted drug delivery in the duodenum. EUDRAGIT® L 100 pH dependent anionic polymer that is soluble at pH above 6.0 for targeted drug delivery in the jejunum. EUDRAGIT® S 100 pH dependent anionic polymer that is soluble at pH above 7.0 for targeted drug delivery in the ileum. EUDRAGIT® E 100 and EUDRAGIT® EPO, pH dependent cationic polymer, soluble up to pH 5.0 and insoluble above pH 5.0.

Additional pH-sensitive materials include poly functional polymers containing multiple groups that become ionized as the pH drops below their pKa. A sufficient quantity of these ionizable groups must be incorporated in the polymer such that in aqueous solutions having a pH below the pKa of the ionizable groups, the polymer dissolves. These ionizable groups can be incorporated into polymers as block copolymers, or can be pendent groups attached to a polymer backbone, or can be a portion of a material used to crosslink or connect polymer chains. Examples of such ionizable groups include polyphosphene, vinyl pyridine, vinyl aniline, polylysine, polyornithine, other proteins, and polymers with substituents containing amino moieties.

pH-sensitive polymers which are relatively insoluble and impermeable at the pH of the stomach, but which are more soluble and permeable at the pH of the small intestine and colon include polyacrylamides, phthalate derivatives such as acid phthalates of carbohydrates, amylose acetate phthalate, cellulose acetate phthalate, other cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinylacetate phthalate copolymer, styrene and maleic acid copolymers, polyacrylic acid derivatives such as acrylic acid and acrylic ester copolymers, polymethacrylic acid and esters thereof, poly acrylic methacrylic acid copolymers, shellac, and vinyl acetate and crotonic acid copolymers.

Preferred pH-sensitive polymers include shellac; phthalate derivatives, particularly cellulose acetate phthalate, polyvinylacetate phthalate, and hydroxypropylmethylcellulose phthalate; polyacrylic acid derivatives, particularly polymethyl methacrylate blended with acrylic acid and acrylic ester copolymers; and vinyl acetate and crotonic acid copolymers.

Anionic acrylic copolymers of methacrylic acid and methylmethacrylate are also particularly useful coating materials for delaying the release of compositions and devices until the compositions and devices have moved to a position in the small intestine which is distal to the duodenum. Copolymers of this type are available from RohmPharma Corp, under the trade names Eudragit-L.R™ and Eudragit-S.R™, are anionic copolymers of methacrylic acid and methylmethacrylate. The ratio of free carboxyl groups to the esters is approximately 1:1 in Eudragit-L.R™ and approximately 1:2 in Eudragit-S.R™. Mixtures of Eudragit-L.R™ and Eudragit-S.R™ can also be used. For coating the compositions and devices of the present invention, these acrylic coating polymers must be dissolved in an organic solvent or mixture of organic solvents. Useful solvents for this purpose are acetone, isopropyl alcohol, and methylene chloride. It is generally advisable to include about 5 to about 20% placticizer in coating formulations of acrylic copolymers. Useful plasticizers are polyethylene glycols, propylene glycols, diethyl phthalate, dibutyl phthalate, castor oil, and triacetin.

Active Ingredients

Active ingredients which can be used in the present invention include food, cosmetic and pharmaceutical active agents. The pharmaceutical active agent can be selected from analgesics, anti-inflammatory agents, anthelmintics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, beta-Blockers, cardiac inotropic agents, corticosteroids, diuretics, anti-parkinsonian agents, gastro-intestinal agents, histamine receptor antagonists, keratolytics, lipid regulating agents, anti-anginal agents, cox-2-inhibitors, leukotriene inhibitors, macrolides, muscle relaxants, nutritional agents, opioid analgesics, protease inhibitors, sex hormones, stimulants, muscle relaxants, anti-osteoporosis agents anti-obesity agents, cognition enhancers, anti-urinary incontinence agents, nutritional oils, anti-benign prostate hypertrophy agents, essential fatty acids, non-essential fatty acids, and mixtures thereof.

A wide variety of therapeutic agents can be used in conjunction with the present invention. The therapeutic agents (e.g. pharmaceutical agents) which may be used in the compositions of the present invention include both water soluble and water insoluble drugs. Examples of such therapeutic agents include antihistamines (e.g., dimenhydrinate, diphenhydramine, chlorpheniramine and dexchlorpheniramine maleate), analgesics (e.g., aspirin, codeine, morphine, dihydromorphone, oxycodone, etc.), naproxyn, diclofenac, indomethacin, flurbiprofen, ketoprofen, piroxican, sulindac, anti-emetics (e.g., metoclopramide), anti-epileptics (e.g., phenytoin, meprobamate and nitrazepam), vasodilators (e.g., nifedipine, papaverine, diltiazem and nicardipine), anti-tussive agents and expectorants (e.g., codeine phosphate), anti-asthmatics (e.g. theophylline and aminophylline), antacids, anti-spasmodics (e.g. atropine, scopolamine), antidiabetics (e.g., insulin), diuretics (e.g., ethacrynic acid, bendrofluazide), anti-hypotensives (e.g., propranolol, clonidine), antihypertensives (e.g., clonidine, methyldopa), bronchodilators (e.g., albuterol), steroids (e.g., hydrocortisone, triamcinolone, prednisone), antibiotics (e.g., tetracycline), antihemorrhoidals, hypnotics, psychotropics, antidiarrheals, mucolytics, sedatives, decongestants, laxatives, vitamins, stimulants (including appetite suppressants such as phenylpropanolamine). The above list is not meant to be exclusive.

Examples of drugs whose efficacious amounts for use in the delivery system of the invention may be determined in this manner include anti-inflammatory agents, including non-steroidal and steroidal anti-inflammatory agents, such as indomethacin, diclofenac, flurbiprofen, aspirin, dexamethasone, budesonide, beclomethasone, flucticasone, tioxocortal, and hydrocortisone; immunosuppressants, such as cyclosporin; bronchodialators, such as salbutamol and theophylline; anti-anginals and anti-hypertensives, such as diltiazem, captopril, nifedipine, isosorbide dinitrate, oxyprenolol; anti-spasmodics, such as cimetropium bromide; anti-neoplastic agents, including methotrexate, tamoxifen, cyclophosphamide, mercaptopurine etoposide; anti-colitis drugs, such as 5-aminosalicylic; and anti-arrhythmia agents, such as quinidine, verapamil, procainamide and lidocaine; protein or peptide drugs, such as insulin, human growth hormone, interleukin-II, interferon, calcitonin, leuprolide, tumor necrosis factor, bone growth factor, melanocyte-stimulating hormone, captopril, somatostatin, somatostatin octapeptide analog, cyclosporin, renin inhibitor, superoxide dismutase; other hormones; vaccines; anti-coagulants, such as heparin or short chain heparin; and anti-migraine drugs, such as ergotamine.

Examples of agents that are useful for colonic delivery include nonsteroidal anti-inflammatory drugs (NSAID) such as diclofenac, flurbiprofen, indomethacin, and aspirin; steroid drugs such as dexamethasone, budesonide, beclomethasone, flucticasone, tioxocortol, and hydrocortisone; contraceptives or steroidal hormones such as estrogen, estradiol and testosterone; immunosuppressants such as cyclosporin; bronchodialators such as theophylline and salbutamol; anti-anginals and anti-hypertensives such as isosorbide dinitrate, isosorbide mononitrate, nitroglycerine, nifedipine, oxyprenolol, diltiazem, captopril, atenolol, benazepril, metoprolol, and vasopril; anti-spasmodic agents such as cimetropium bromide; anti-colitis agents such as 5-aminosalicylic acid; anti-arrhythmia agents such as quinidine, verapamil, procainamide, and lidocaine; anti-neoplastic agents such as methotrexate, tamoxifen, cyclophosphamide, mercaptopurine, and etoposide; protein or peptide drugs such as insulin, human growth hormone, interleukin-II, interferon, calcitonin, leuprolide, tumor necrosis factor, bone growth factor, melanocyte-stimulating hormone, captopril, somatostatin, somatostatin octapeptide analog, cyclosporin, renin inhibitor, superoxide dismutase, other hormones and vaccines; anticoagulants such as heparin or short chain heparin, anti-migraine drugs such as ergotomine; glibenclamide; 5-hydroxytryptamine type_{1A} receptor agonist gepiron; 5HT₃ antagonist ondasteron; metkephamid; menthol; antibiotics such as neomycin, beta-lactams such as ampicillin and amoxicillin, cephalosporins such as cephalexin and cloxacillin, and macrolides such as erythromycin; and PGE₁ analogues for protecting the gastroduodenal mucosa from NSAID injury, such as misoprostol. Protein drugs, such as LH-RH and insulin, may survive longer and be absorbed better from the colon than from the small intestine. Other drugs have been shown to possess colonic absorption, such as diclofenac, quinidine, theophylline, isosorbide dinitrate, nifedipine, oxprenolol, metoprolol, glibenclamide, 5-hydroxytryptamine type_{1A} receptor agonist gepiron, 5HT₃ antagonist ondasteron, metkephamid, menthol, benazepril (ACE inhibitor).

Examples of drugs that are useful for treating various other regions of the alimentary canal are as follows: Gastro Esophagal Reflux Disease—H2 receptor antagonists (e.g., Tagamet, Zantac), proton pump inhibitors (e.g., Omeprazole); Candida esophagitis—nystatin or clotrimazole; Duodenal Ulcer—H2 receptor agonists, prostaglandins (e.g., Cytotec, Prostin), proton pump inhibitors—(e.g., Prilosec, Omeprazole, Sucralfate); Pathological Hypersecretory Conditions, Zollinger-Ellison Syndrome—H2 receptor agonists; Gastritis—H2 receptor agonists, PGE₁ analogs for protecting the gastroduodenal mucosa from NSAID injury such as misoprostol, GHR-IH drugs for treating pancreatitis, such as somatostatin, and anti-spasmodic drugs for treating local spasmolytic action such as cimetropium bromide.

The therapeutic benefits of the delivery system depend upon its ability to delivery efficacious levels of drugs to a specific site in the gastrointestinal tract. This allows the local treatment of diseases including, but not limited to, ulcerative colitis, Crohn's disease, colon carcinoma, esophagitis, Candida esophagitis, duodenal ulcers, gastric ulcers, Zollinger-Ellison Syndrome (gastrinoma), gastritis, chronic constipation, pancreatitis, local spasms, local infections, parasites, and other changes within the gastrointestinal tract due to effects of systemic disorders (e.g., vascular inflammatory, infectious and neoplastic conditions).

Active components may be added using any of the known methods described in the prior art, and such addition may be carried out during and/or after the production of the matrix composition. Typical active components may include, but are not limited to, a therapeutic substance or a pharmaceutically active agent such as a drug, a non-therapeutic substance such as a cosmetic substance, a local or general anesthetic or pain killer, or an opiate, a vaccine, an antigen, a microorganism, a sterilizing substance, a contraceptive composition, a protein or peptide such as insulin, an insecticide, a herbicide, a hormone such as a growth hormone or a seed germination hormone, a steroid, a toxin, or a marker substance. A non-limiting list of possible active components includes hydrochlorothiazide, acetazolamide, acetylsalicyclic acid, allopurinol, alprenolol, amiloride, antiarrhythmics, antibiotics, antidiabetics, antiepileptics, anticoagulants, antimycotics, atenolol, bendroflumethiazide, benzbromarone, benzthiazide, betamethasone, bronchodilators, buphenine, bupranolol, chemotherapeutics, chlordiazepoxide, chlorquine, chloro thiazide, chlorpromazine, chlortalidone, clenbuterol, clomipramine, clonidine, co-dergocrine, cortisone, dexamethasone, dextropropoxyphene, diazepam, diazoxide, diclofenac, diclofenamide, digitalisglycoside, dihydralazine, dihydroergotamine, diltiazem, iron salt, ergotamine, ethacrynic acid, ethinylestradiol, ethoxzolamide, fenoterol, fludrocortisone, fluphenazine, fluorosemide, gallopamil, guanethidine, hormones, hydrochlorothiazide, hydrocortisone, hydroflumethiazide, immunosuppresives, ibuprofen, imipramine, indomethacine, coronartherapeutics, levodopa, lithium salt, magnesium salt, medroxyprogesteron acetate, manadione, methaqualone, 8-methoxypsoralen, methylclothiazide, methyldopa, methylprednisolone, methyltestosterone, methylthiouracil, methylxanthine, metipranolol, molsidomine, morphine, naproxen, nicergline, nifedipine, norfenefrine, oxyphenbutazone, papaverine, parmathasone, pentobarbital, perphenazine, phenobarbital, phenylbutazone, phytomenadione, pirenzepine, polythiazide, prazosine, prednisolone, prednisone, probenecid, propranolol, propylthiouracil, rescinnamine, reserpine, secbutabarbital, secobarbital, spironolactone, sulfasalazine, sulfonamide, testosterone, thioridazine, triamcinolon, triamteren, trichloromethiazide, trifluoperazine, trifluopromazine, tuberculostatic, verapamil, virustatics, zytostatics, bromocriptine, bromopride, carbidopa, carbocromen, quinine, chlorprothixene, cimetidine, clofibrat, cyclizine, desipramine, disulfiram, domperidone, doxepine, fenbufen, flufenamine acid, flunarizine, gemfibrocil, haloperidol, ketoprofen, labetalol, lorazepam, mefenamine acid, melperone, metoclopramide, nortriptyline, noscapine, oxprenolol, oxymetholone, pentazocine, pethidine, stanozolol, sulindac, sulpiride, tiotixen.

Suitable active ingredients are those which exert a local physiological effect, as well as those which exert a systemic effect, either following penetrating the mucosa or—in the case of oral administration—following transport to the gastro-intestinal tract with saliva. The bioadhesive dosage forms prepared from the compositions according to the present invention are particularly suitable for active ingredients which exert their activity during an extended period of time. Examples thereof are: analgesic and anti-inflammatory drugs (NSAIDs, acetyl salicylic acid, diclofenac sodium, ibuprofen, indomethacin, ketoprofen, meclofenamate sodium, mefenamic acid, naproxen sodium, paracetamol, piroxicam, tolmetin sodium); anti-arrhythmic drugs (procainamide HCl, quinidine sulphate, verapamil HCl); antibacterial agents (amoxicillin, ampicillin, benzathine penicillin, benzylpenicillin, cefaclor, cefadroxil, cephalexin, chloramphenicol, ciprofloxacin, clavulanic acid, clindamycin HCl, doxyxycline HCl, erythromycin, flucloxacillin sodium, kanamycin sulphate, lincomycin HCl, minocycline HCl, nafcillin sodium, nalidixic acid, neomycin, norfloxacin, ofloxacin, oxacillin, phenoxymethyl-penicillin potassium); anti-coagulants (warfarin); antidepressants (amitriptyline HCl, amoxapine, butriptyline HCl, clomipramine HCl, desipramine HCl, dothiepin HCl, doxepin HCl, fluoxetine, gepirone, imipramine, lithium carbonate, mianserin HCl, milnacipran, nortriptyline HCl, paroxetine HCl); anti-diabetic drugs (glibenclamide); anti fungal agents (amphotericin, clotrimazole, econazole, fluconazole, flucytosine, griseofulvin, itraconazole, ketoconazole, miconazole nitrate, nystatin); antihistamines (astemizole, cinnarizine, cyproheptadine HCl, flunarizine, oxatomide, promethazine, terfenadine); anti-hypertensive drugs (captopril, enalapril, ketanserin, lisinopril, minoxidil, prazosin HCl, ramipril, reserpine); anti-muscarinic agents (atropine sulphate, hyoscine); antivirals (acyclovir, AZT, ddC, ddI, ganciclovir, loviride, tivirapine, 3TC, delavirdine, indinavir, nelfinavir, ritonavir, saquinavir); sedating agents (alprazolam, buspirone HCl, chlordiazepoxide HCl, chlorpromazine, clozapine, diazepam, flupenthixol HCl, fluphenazine, flurazepam, lorazepam, mazapertine, olanzapine, oxazepam, pimozide, pipamperone, piracetam, promazine, risperidone, selfotel, seroquel, sulpiride, temazepam, thiothixene, triazolam, trifluperidol, ziprasidone); anti-stroke agents (lubeluzole, lubeluzole oxide, riluzole, aptiganel, eliprodil, remacemide); anti-migraine drugs (alniditan, sumatriptan); beta-adrenoceptor blocking agents (atenolol, carvedilol, metoprolol, nebivolol, propanolol); cardiac inotropic agents (digitoxin, digoxin, milrinone); corticosteroids (beclomethasone dipropionate, betamethasone, dexamethasone, hydrocortisone, methylprednisolone, prednisolone, prednisone, triamcinolone); disinfectants (chlorhexidine); diuretics (acetazolamide, frusemide, hydrochlorothiazide, isosorbide); anti-Parkinsonian drugs (bromocryptine mesylate, levodopa, selegiline HCl); enzymes; essential oils (anethole, anise oil, caraway, cardamom, cassia oil, cineole, cinnamon oil, clove oil, coriander oil, dementholised mint oil, dill oil, eucalyptus oil, eugenol, ginger, lemon oil, mustard oil, neroli oil, nutmeg oil, orange oil, peppermint, sage, spearmint, terpineol, thyme); gastro-intestinal agents (cimetidine, cisapride, clebopride, diphenoxylate HCl, domperidone, famotidine, lansoprazole, loperamide HCl, loperamide oxide, mesalazine, metoclopramide HCl mosapride, olsalazine, omeprazole, ranitidine, rabeprazole, ridogrel, sulphasalazine); haemostatics (aminocaproic acid); lipid regulating agents (lovastatin, pravastatin, probucol, simvastatin); local anaesthetics (benzocaine, lignocaine); opioid analgesics (buprenorphine HCl, codeine, dextromoramide, dihydrocodeine); parasympathomimetics (galanthamine, neostigmine, physostymine, tacrine, donepezil, ENA 713 (exelon), xanomeline); vasodilators (amlodipine, buflomedil, amyl nitrite, diltiazem, dipyridamole, glyceryl trinitrate, isosorbide dinitrate, lidoflazine, molsidomine, nicardipine, nifedipine, oxpentifylline, pentaerythritol tetranitrate).

Active agents which can be incorporated into the matrix for delivery include therapeutic or prophylactic agents. These can be proteins or peptides, sugars, oligosaccharides, nucleic acid molecules, or other synthetic or natural agents. The agents may be labeled with a detectable label such as a fluorescent label or an enzymatic or chromatographically detectable agent.

Preferred drugs include antibiotics, antivirals, vaccines, vasodilators, vasoconstrictors, immunomodulatory compounds, including steroids, antihistamines, and cytokines such as interleukins, colony stimulating factors, tumor necrosis factor and interferon (alpha, beta, gamma), oligonucleotides including genes and antisense, nucleases, bronchodilators, hormones including reproductive hormones, calcitonin, insulin, erthropoietin, growth hormones, and other types of drugs such as Antiban™.

The matrix composition and articles formed of the matrix composition of the present invention can also include encapsulated active ingredients in the form of micro-spheres, micro-particles, nano-spheres, hydrophobic nano-spheres encapsulated in a water, or pH sensitive micro-spheres.

Water Sensitive Micro-Spheres

Water sensitive micro-spheres can be incorporated in the compositions and articles of the present invention by mixing the microspheres with a water sensitive material before dispersing the microspheres in the matrix composition. In an embodiment of the present invention, water sensitive micro-spheres comprising the active ingredients can be used to replace the water sensitive materials used in the matrix composition.

Water-sensitive materials for use to encapsulate active ingredients in the present invention comprises of water soluble and water dispersible natural oligomers, synthetic oligomers, natural polymers, synthetic polymers and copolymers, starch derivatives, oligosaccharide, polysaccharides, hydrocolloids, natural gums, proteins, and mixtures thereof.

Suitable water soluble materials include xylose, ribose, glucose, mannose, galactose, fructose, dextrose, polydextrose, sucrose, maltose, or corn syrup solids, palatin, sorbitol, xylitol, mannitol, maltitol, lactitol, xanthan, maltodextrin, galactomanan or tragacanth, and mixtures thereof. Water sensitive materials include oligosaccharides and hydrocolloids.

Examples of synthetic water sensitive polymers which are useful for the invention include polyvinyl pyrrolidone, water soluble celluloses, polyvinyl alcohol, ethylene maleic anhydride copolymer, methylvinyl ether maleic anhydride copolymer, acrylic acid copolymers, anionic polymers of methacrylic acid and methacrylate, cationic polymers with dimethyl-aminoethyl ammonium functional groups, polyethylene oxides, water soluble polyamide or polyester.

Examples of water soluble hydroxyalkyl and carboxyalkyl celluloses include hydroxyethyl and carboxymethyl cellulose, hydroxyethyl and carboxyethyl cellulose, hydroxymethyl and carboxymethyl cellulose, hydroxypropyl carboxymethyl cellulose, hydroxypropyl methyl carboxyethyl cellulose, hydroxypropyl carboxypropyl cellulose, hydroxybutyl carboxymethyl cellulose, and the like. Also useful are alkali metal salts of these carboxyalkyl celluloses, particularly and preferably the sodium and potassium derivatives.

The polyvinyl alcohol useful in the practice of the invention is partially and fully hydrolyzed polyvinyl acetate, termed “polyvinyl alcohol” with polyvinyl acetate as hydrolyzed to an extent, also termed degree of hydrolysis, of from about 75% up to about 99%. Such materials are prepared by means of any of Examples I-XIV of U.S. Pat. No. 5,051,222 issued on Sep. 24, 1991, the specification for which is incorporated by reference herein.

Polyvinyl alcohol useful for practice of the present invention is Mowiol® 3-83, having a molecular weight of about 14,000 Da and degree of hydrolysis of about 83%, Mowiol® 3-98 and a fully hydrolyzed (98%) polyvinyl alcohol having a molecular weight of 16,000 Da commercially available from Gehring-Montgomery, Inc. of Warminister Pa. Other suitable polyvinyl alcohols are: AIRVOL® 205, having a molecular weight of about 15,000-27,000 Da and degree of hydrolysis of about 88%, and VINEX® 1025, having molecular weight of 15,000-27,000 Da degree of hydrolysis of about 99% and commercially available from Air Products & Chemicals, Inc. of Allentown, Pa.; ELVANOL® 51-05, having a molecular weight of about 22,000-26,000 Da and degree of hydrolysis of about 89% and commercially available from the Du Pont Company, Polymer Products Department, Wilmington, Del.; ALCOTEX® 78 having a degree of hydrolysis of about 76% to about 79%, ALCOTEX® F88/4 having a degree of hydrolysis of about 86% to about 88% and commercially available from the Harlow Chemical Co. Ltd. Of Templefields, Harlow, Essex, England CM20 2BH; and GOHSENOL® GL-03 and GOHSENOL® KA-20 commercially available from Nippon Gohsei K.K., The Nippon Synthetic Chemical Industry Co., Ltd., of No. 9-6, Nozaki Cho,Kita-Ku, Osaka, 530 Japan.

Suitable polysaccharides are polysaccharides of the non-sweet, coloidally-soluble types, such as natural gums, for example, gum arabic, starch derivates, dextrinized and hydrolyzed starches, and the like. A suitable polysaccharide is a water dispersible, modified starch commercially available as Capule®, N-Lok®, Hi-Cap™ 100 or Hi-Cap™ 200 commercially available from the National Starch and Chemical Company of Bridgewater, N.J.; Pure-Cote™, commercially available from the Grain Processing Corporation of Muscatine, Iowa. In the preferred embodiment the natural gum is a gum arabic, commercially available from TIC Gums Inc. Belcamp, Midland. Suitable hydrocolloids are xanthan, maltodextrin, galactomanan or tragacanth, preferably maltodextrins such as Maltrin™ M100, and Maltrin™ M150, commercially available from the Grain Processing Corporation of Muscatine, Iowa.

In one embodiment, the water sensitive micro-spheres can be bioadhesive. Bioadhesive micro-sphere can be created by incorporating a bioadhesive material into the micro-sphere matrix.

The water-sensitive micro-spheres of the present invention comprising active ingredients can be prepared by the steps of (1) forming an aqueous phase of the moisture sensitive materials (either a single material or mixture of several materials); (2) emulsifying the active ingredients in the aqueous phase; and (3) removing moisture to create free-flowing powder. For example, moisture can be removed by spray drying droplets of emulsion. Spray drying is well known in the art and been used commercially in many applications, including foods where the core material is a flavoring oil and cosmetics where the core material is a fragrance oil, as described in Cf. Balassa, “Microencapsulation in the Food Industry”, CRC Critical Review Journal in Food Technology, July 1971, pp 245-265; Barreto, “Spray Dried Perfumes for Specialties, Soap and Chemical Specialties”, December 1966; Maleeny, Spray Dried Perfumes, Soap and San Chem, January 1958, pp. 135 et seq.; Flinn and Nack, “Advances in Microencapsulation Techniques”, Batelle Technical Review, Vo. 16, No. 2, pp. 2-8 (1967); U.S. Pat. Nos. 5,525,367; and 5,417,153 which are incorporated herein as references.

The micro-spheres of the present invention preferably have size of from about 0.5 micron to about 300 microns, more preferably from about 1 micron to about 200 microns, most preferably from about 2 microns to about 30 microns. The present invention preferably has minimal active agents on the surface of the spheres, preferably less than 1%.

Hydrophobic Nano-Spheres Encapsulated in Water Sensitive Micro-Spheres

Multi component carrier systems, comprising of solid hydrophobic nano-spheres encapsulated in a moisture, water, or pH sensitive micro-sphere, can also be incorporated in the compositions and devices of the present invention by mixing them with the water sensitive materials before dispersing them in the composition. These multi component systems provides moisture-triggered release of the actives that are encapsulated in the micro-sphere matrix, as well as, prolong release of the actives encapsulated that are encapsulated in the nano-sphere matrix over an extended period of time. The surface properties of the nano-spheres may be modified to enhance the affinity of the nano-spheres for a particular residue expressed on a cell surface or their affinity for a cell surface protein or receptor. Active ingredients can be incorporated in the hydrophobic nano-spheres, in the water, or pH sensitive micro-spheres, or in both the nano and micro-spheres. The deposition of the nano-spheres onto the target surface is improved by optimizing particle size to ensure entrainment of the particles within target surface and by modifying their surface to enhance the affinity of the nano-spheres for a particular residue expressed on a cell surface or their affinity for a cell surface protein or receptor to maximize interaction between the particles and the target surface.

With respect to the interaction between the particles and the target surface, various chemical groups and bioadhesive materials can be incorporated in the nano-spheres structure, depending on the target surface. A cationic surface active agent will create positively charged nano-spheres; an anionic surface active agent will create negatively charged nano-spheres; a nonionic surface active will create neutral charged nano-spheres; and a zwitterionic surface active agent will create a variable charged nano-spheres.

In one embodiment, the nano-spheres of the present invention are bioadhesive. Bioadhesive nano-sphere can be created by incorporating a bioadhesive material into the solid hydrophobic matrix of the nano-spheres, by incorporating bioadhesive material in the pH sensitive micro-sphere matrix, or by using a bioadhesive material in the nano-sphere matrix in conjunction with bioadhesive material in the micro-sphere matrix.

These multi component systems are in the form of free-flowing, powder, having the advantages of:

(i) protection of the active ingredients, during storage, or until needed and reaches the target site;

(ii) water, or pH triggered release of the first said active ingredient and the nano-spheres comprising the second said active ingredient in response to moisture or in response to change in pH in the system proximate environment, and,

(iii) site specific targeted delivery and enhanced deposition of active ingredients, onto the target surface;

(iv) enhanced bioavelability of active ingredients encapsulated in the nano-spheres; and

(v) prolonged release of active ingredients, over an extended period of time.

The method for producing the multi component controlled release system including active ingredients that comprises the steps of:

(i) incorporating the active ingredients into the solid hydrophobic nano-spheres;

(ii) forming an aqueous mixture comprising of one or more active agents, the nano-spheres, and a water, or pH or sensitive materials; and

(iii) spray drying the mixture to form a dry powder composition.

The process for producing the multi component controlled release system including the active ingredients that comprises the steps of:

(ix) heating hydrophobic materials to a temperature above the melting point of the materials to form a melt;

(x) dissolving or dispersing the first active agent into the melt, and optionally a targeting material;

(xi) dissolving or dispersing a second active agent, the water or pH sensitive material, and optionally a targeting material, in the aqueous phase;

(xii) heating the composition to above the melting temperature of the hydrophobic materials;

(xiii) mixing the hot melt with the aqueous phase to form a dispersion;

(xiv) high shear homogenization of the dispersion at a temperature above the melting temperature until a homogeneous fine dispersion is obtained having a sphere size of from about 1 micron to about 2 microns;

(xv) cooling the dispersion to ambient temperature; and

(xvi) spray drying the emulsified mixed suspension to form a dry powder composition.

The hydrophobic matrix sustains the diffusion rate of the pharmacotherapeutic active ingredients, through the nano-spheres and enables them to be released onto the target site over an extended period of time. The micro-spheres have an average sphere size in the range from about 20 microns to about 100 microns. The nano-sphere have an average sphere size in the range from about 0.01 micron to about 5 microns and having a melting point in the range from about 30 degrees C. to about 90 degrees C.

Nano-spheres formed of a hydrophobic material provide a controlled release system in order to release the active agent over an extended period of time by molecular diffusion. Active agents in the hydrophobic matrix of the nano-spheres can be released by transient diffusion. The theoretical early and late time approximation of the release rate of the active ingredients dissolved in the hydrophobic matrix of the nano-spheres can be calculated from the following equations:

Early time approximation

$$\begin{matrix}( {m}_{t} / {m}_{s ec} ) < 0.4 \\ \frac{{M}_{t}}{{M}_{∞}} = 4 {( \frac{{D}_{p} t}{Π {r}^{2}} )}^{1 / 2} - \frac{{D}_{p} t}{ {r}^{2}} & ( 1 ) \\ \frac{ⅆ {M}_{t} / {M}_{∞}}{ⅆ t} = 2 {( \frac{{D}_{p}}{Π {r}^{2} t} )}^{1 / 2} - \frac{{D}_{p}}{ {r}^{2}} & ( 2 )\end{matrix}$$

Late time approximation

$$\begin{matrix}( {m}_{t} / {m}_{∞} ) > 0.6 \\ \frac{{M}_{t}}{{M}_{∞}} = 1 - \frac{4}{{( 2.405 )}^{2}} exp ( \frac{- {( 2.405 )}^{2} {D}_{p} t}{ {r}^{2}} ) & ( 3 ) \\ \frac{ⅆ {M}_{t} / {M}_{∞}}{ⅆ t} = 1 - \frac{4 {D}_{p}}{{r}^{2}} exp ( \frac{- {( 2.405 )}^{2} {D}_{p} t}{ {r}^{2}} ) & ( 4 )\end{matrix}$$

wherein:

r is the radius of the cylinder,

m ∞ is the amount fragrance released from the controlled release system after infinite time;

mₜ is the amount fragrance released from the controlled release system after time t; and

Dₚ is the diffusion coefficient of the fragrance or aroma chemical in the matrix.

The release rate for releasing the active agents from the hydrophobic nano-spheres is typically slower than the release rate for releasing active agent from the water or pH sensitive matrix. The active agents can be selected to be incorporated into either the hydrophobic nano-spheres or the water or pH sensitive matrix depending on the desired time for release of the active agents. For example, the water or pH sensitive matrix formed in accordance with the present invention can release the first active agent at a predetermined pH to provide a “burst” with continued release of the first active agent and nano-spheres formed in accordance with the present invention can release the active agent depending on the release rate from an initial time such as within few days, up to a period of few weeks.

Diagnostic Applications and Gastrointestinal Imaging

The system of the present invention is also useful for diagnostic purposes, such as site-specific delivery of x-ray contrast agents, such as barium sulfate, siatrizoate sodium, iodine containing contrast agents, ultrasound contrast agents, contrast or enhancement agents for Magnetic Resonance Imaging, Tomography, or Positron Emission agents. The system is further useful for the delivery of monoclonal antibody markers for tumors.

The composition and articles of the present invention can contain imaging agents that can be used in vascular imaging, as well as in applications to detect liver and renal diseases, in cardiology applications, in detecting and characterizing tumor masses and tissues, and in measuring peripheral blood velocity.

Barium sulfate suspension is the universal contrast medium used for examination of the upper gastrointestinal tract, as described by D. Sutton, Editor, A Textbook of Radiology and Imaging, Volume 2, Churchill Livingstone, London (1980), even though it has undesirable properties, such as unpalatability and a tendency to precipitate out of solution.

Several properties of the system are advantageous in diagnostic and gastrointestinal imaging, such as: (a) particle size: the rate of sedimentation is proportional to particle size (i.e., the finer the particle, the more stable the suspension); (b) non-ionic medium because charges on the barium sulfate particles influence the rate of aggregation of the particles, and aggregation is enhanced in the presence of the gastric contents; and (c) solution pH: suspension stability is best at pH 5.3, however, as the suspension passes through the stomach, it is inevitably acidified and tends to precipitate.

The encapsulation of barium sulfate in the matrix composition and articles found of the matrix composition can help in coating, preferentially, the gastric mucosa in the presence of excessive gastric fluid. With bioadhesiveness targeted to more distal segments of the gastrointestinal tract, it can also provide a kind of wall imaging not easily obtained otherwise.

The double contrast technique, which utilizes both gas and barium sulfate to enhance the imaging process, uses a proper coating of the mucosal surface. To achieve a double contrast, air or carbon dioxide must be introduced into the patient's gastrointestinal tract. This is typically achieved via a nasogastric tube to provoke a controlled degree of gastric distension. Comparable results can be obtained by the release of individual gas bubbles in a large number of individual adhesive microspheres of the present invention and this imaging process can apply to intestinal segments beyond the stomach.

An in vivo method for evaluating bioadhesion uses encapsulation of a radio-opaque material, such as barium sulfate, or both a radio-opaque material and a gas-evolving agent, such as sodium carbonate, within a bioadhesive polymer. After oral administration of this radio-opaque material, its distribution in the gastric and intestinal areas is examined using image analysis.

Processing of the Matrix Composition for Forming Articles

The compositions can be easily molded and processed into an article of any predetermined shape or form. The articles remain stable upon storage under a variety of conditions. The articles disintegrate at a controllable kinetic mechanism by surface and/or bulk erosion into small pieces that are non-toxic and readily eliminated by the body in vivo.

The compositions can be utilized to prepare a wide range of articles such as an implant. For example, the matrix composition can be melted, mixed with a substance to be delivered, and then solidified by cooling. The melt fabrication processes use matrix compositions having a melting point that is below the temperature at which the substance to be delivered degrades or become reactive.

Methods of producing articles such as particulates and implants also include granulation, spheronization, spray congealing, solvent casting, extrusion and molding. These methods can be used to form articles of micro-implants such as microparticles, microspheres, and microcapsules encapsulating drug to be released, capsules, tablets, slabs or sheets, films, tubes, medical devices and other structures. The micro-implants can be used for infusion or injection. The micro-implants can be used as dental bone implants.

Articles of the present invention can be formed by extrusion. Extrusion is a well-known method of applying pressure to a damp or melted composition until it flows through an orifice or a defined opening. The extrudable length varies with the physical characteristics of the material to be extruded, the method of extrusion, and the process of manipulation of the particles after extrusion. Various types of extrusion devices can be employed, such as screw, sieve and basket, roll, and ram extruders. Components of the matrix composition of the present invention can be melted and extruded with a continuous, solvent free extrusion process, with or without inclusion of additives by conventional method.

Articles of the present invention can be formed of spheronization. Spheronization is the process of converting material into spheres. A shape having the lowest surface area to volume ratio. Conventional spheronization techniques begin with damp extruded particles. The extruded particles are broken into uniform lengths instantaneously and gradually transformed into spherical shapes. In addition, powdered raw materials, which require addition of either liquid or material from a mixer, can be processed in an air-assisted spheronizer.

Articles formed of the matrix composition and articles of the matrix composition of the present invention can be formed by spray congealing. Spray congealing is method that is generally used in changing the structure of the materials such as to obtain free flowing powders from liquids and to provide pellets ranging in size from about 0.25 to 2.0 mm. Spray congealing is process in which a substance of interest is allowed to melt, disperse, or dissolve in a hot melt of other additives, and is then sprayed into an air chamber wherein the temperature is below the melting point of the formulation components, to provide spherical congealed pellets. The air removes the latent heat of fusion. The temperature of the cooled air used depends on the freezing point of the product. The particles are held together by solid bonds formed from the congealed melts. Due to the absence of solvent evaporation in most spray congealing processes, the particles are generally non porous and strong, and remain intact upon agitation. The characteristics of the final congealed product depend in part on the properties of the additives used. The rate of feeding and inlet/outlet temperatures are adjusted to ensure congealing of the atomized liquid droplet. The feed should have adequate viscosity to ensure homogeneity. The conversion of molten feed into powder is a single, continuous step. Proper atomization and a controlled cooling rate are critical to obtain high surface area, uniform and homogeneous congealed pellets. Adjustment of these parameters is readily achieved by one skilled in the art. The spray congealing method is particularly suitable for heat labile substances, since ambient temperature is used to dry, and for moisture sensitive substances, since non-aqueous compositions can be utilized. Spray congealing is similar to spray drying, except that no solvent is utilized. Spray congealing is a uniform and rapid process, and is completed before the product comes in contact with any equipment surface. Most additives that are solid at room temperature and melt without decomposition are suitable for this method. Conventional spray dryers operating with cool inlet air have been used for spray congealing. Several methods of atomization of molten mass can be employed, such as pressure, or pneumatic or centrifugal atomization. For persons skilled in the spray congealing art, it is well known that several formulation aspects, such as matrix materials, viscosity, and processing factors, such as temperature, atomization and cooling rate affect the quality (morphology, particle size distribution, polymorphism and dissolution characteristics) of spray congealed pellets. The spray congealed particles may be used in tablet granulation form, encapsulation form, or can be incorporated into a liquid suspension form.

A cryopelletization procedure can be used in the present invention to allow conversion of a molten mass, aqueous solution, or suspension into solid, bead-like particles to form the matrix composition or materials formed of the matrix composition. The molten mass solutions or suspensions are dripped by means of an appropriately designed device into liquid nitrogen. The production of small drops and liquid nitrogen cooling permit very rapid and uniform freezing of the material processed. The pellets are further dried in conventional freeze dryers. Cryopelletization can also be carried out under aseptic conditions for sterile processing. The most critical step producing spherical particles by globulization is the droplet formation. Droplet formation is influenced by formulation related variables, such as the nature of the active ingredient and additives, viscosity, total solid content, surface tension, etc. In addition, equipment design and processing variable also play an important role. One skilled in the art can readily balance the various factors to produce a satisfactory product.

The use of extrusion and spray congealing for producing solid compositions of the pharmaceutical compositions of the present invention are described in detail in U.S. Pat. Nos. 5,965,161 and 5,539,000 respectively, the disclosures of which are incorporated herein by reference.

Articles can be formed of the matrix composition in one of several ways. For example, the polymer can be melted, mixed with the substance to be delivered, and then solidified by cooling. Such melt fabrication processes require polymers having a melting point that is below the temperature at which the substance to be delivered and polymer degrade or become reactive. Alternatively, the article can be prepared by solvent casting, where the polymer is dissolved in a solvent, and the substance to be delivered dissolved or dispersed in the polymer solution. The solvent is then evaporated, leaving the substance in the polymeric matrix. Solvent casting requires that the polymer be soluble in organic solvents and that the drug to be encapsulated be soluble or dispersible in the solvent. Similar articles can be made by phase separation or emulsification or even spray drying techniques. In still other methods, a powder of the polymer is mixed with the drug and then compressed to form an article such as an implant.

Methods of producing articles of the present invention also include granulation, extrusion, and spheronization. A dry powder blend is produced including the desired excipients and microspheres. The dry powder is granulated with water or other non-solvents for microspheres such as oils and passed through an extruder forming “strings” or “fibers” of wet massed material as it passes through the extruder screen. The extrudate strings are placed in a spheronizer which forms spherical particles by breakage of the strings and repeated contact between the particles, the spheronizer walls and the rotating spheronizer base plate. The implants are dried and screened to remove aggregates and fines.

The invention can be further illustrated by the following examples thereof, although it will be understood that these examples are included merely for purposes of illustration and are not intended to limit the scope of the invention unless otherwise specifically indicated. All percentages, ratios, and parts herein, in the Specification, Examples, and Claims, are by weight and are approximations unless otherwise stated.

### PREPARATION OF MATRIX COMPOSITIONS

### EXAMPLE 1

Compositions according to the present invention were prepared as follows. The specific components used are detailed in compositions I to III. The wax was melted at about ten degrees above the melting point. In a separate vessel the fat was also melted at about ten degrees above the melting point of the wax. The appropriate surfactants are added to the fat melt, while homogenizing the melt at a temperature about ten degrees above the melting point of the wax. The wax and fat melts are combined and the water-soluble materials and the active ingredients are added to the melt slowly while homogenizing the mixture.

### Composition I

A pharmaceutical composition was prepared according to the method of Example 1, in the form of particle processed by the cryopelletization procedure that allows conversion of the molten mass into solid, bead-like particles. The pharmaceutical composition is formed from a mixture of glyceride and fat (Glyceryl Monostearate and Myverol 18-07), a wax mixture (Carnauba wax and Candelilla wax) an active ingredient (Calcium sulfate), a mixture of water soluble materials (Hi-Cap 100 and Lactitol), and a mixture of a mixture of non-ionic hydrophilic surfactants (Cremophor RH-40 and Tween 20). The components and their amounts were as follows:

| Component | Weight (gr) | % (w/w) |
|---|---|---|
| Candelilla wax | 150 | 15.0 |
| Carnauba wax | 200 | 20.0 |
| Glyceryl monostearate | 50 | 5.0 |
| Myverol 18-07 | 5 | 0.5 |
| Cremophor RH-40 | 5 | 0.5 |
| Tween 20 | 50 | 5.0 |
| Hi-Cap 100 | 10 | 1.0 |
| Lactitol | 30 | 3.0 |
| Calcium sulfate | 500 | 50.0 |

### Composition II

A pharmaceutical composition was prepared according to the method of Example 1, in the form of particle processed by the cryopelletization procedure that allows conversion of the molten mass into solid, bead-like particles. The pharmaceutical composition is formed from a fat of Glyceryl Monostearate, a wax (Carnauba wax) an active ingredient (Calcium sulfate), a water soluble materials (Lactitol), and a mixture of a non-ionic surfactant (Tween 20). The components and their amounts were as follows:

| Component | Weight (gr) | % (w/w) |
|---|---|---|
| Carnauba wax | 400 | 40.0 |
| Glyceryl monostearate | 50 | 5.0 |
| Tween 20 | 40 | 4.0 |
| Lactitol | 10 | 1.0 |
| Calcium sulfate | 500 | 50.0 |

### Composition III

A pharmaceutical composition was prepared according to the method of Example 1, in the form of particle processed by the cryopelletization procedure that allows conversion of the molten mass into solid, bead-like particles. The pharmaceutical composition is formed from a fat (Myverol 18-07), a wax mixture (Carnauba wax and Candelilla wax) an active ingredient (Calcium sulfate), a mixture of water soluble materials (Maltrin 180), and a mixture of a non-ionic surfactant (Cremophor RH-40). The components and their amounts were as follows:

| Component | Weight (gr) | % (w/w) |
|---|---|---|
| Candelilla wax | 250 | 25.0 |
| Carnauba wax | 200 | 20.0 |
| Myverol 18-07 | 10 | 1.0 |
| Cremophor RH-40 | 30 | 3.0 |
| Maltrin 180 | 10 | 1.0 |
| Calcium sulfate | 500 | 50.0 |

### PREPARATION OF A PHARMACEUTICAL COMPOSITION FOR AN IMAGING APPLICATION

### EXAMPLE 2

Compositions according to the present invention were prepared as follows. The specific components used are detailed in compositions VI to VI. The wax was melted at about ten degrees above the melting point. In a separate vessel the fat was also melted at about ten degrees above the melting point of the wax. The appropriate surfactants are added to the fat melt, while homogenizing the melt at a temperature about ten degrees above the melting point of the wax. The wax and fat melts are combined and the water-soluble materials and the active ingredients are added to the melt slowly while homogenizing the mixture.

### Composition IV

A pharmaceutical composition for imaging applications was prepared according to the method of Example 2, in the form of tablets and other predetermined shape processed by molding the molten mass into molds of various shapes and cooling the molded mass. The pharmaceutical composition is formed from a fat (Glyceryl Monostearate), a wax mixture (Carnauba wax and Candelilla wax), a water soluble materials (Maltrin 150), and a non-ionic hydrophilic surfactant (Tween 20). The components and their amounts were as follows:

| Component | Weight (gr) | % (w/w) |
|---|---|---|
| Candelilla wax | 150 | 15.0 |
| Carnauba wax | 200 | 20.0 |
| Glyceryl monostearate | 100 | 10.0 |
| Tween 20 | 150 | 15.0 |
| Maltrin 150 | 400 | 40.0 |

The formed article can be used a carrier system for other imaging devices.

### Composition V

A pharmaceutical composition for imaging applications was prepared according to the method of Example 2, in the form of tablets and other predetermined shapes processed by molding the molten mass into several molds of various shapes and cooling it. The pharmaceutical composition is formed from a of fat (Glyceryl Monostearate), a wax (Carnauba wax) an active ingredient (Barium sulfate), a water soluble materials (Lactitol), and a mixture of a non-ionic surfactant (Tween 20). The components and their amounts were as follows:

| Component | Weight (gr) | % (w/w) |
|---|---|---|
| Carnauba wax | 300 | 30.0 |
| Beeswax | 50 | 5.0 |
| Glyceryl monostearate | 100 | 10.0 |
| Tween 20 | 150 | 15.0 |
| Lactitol | 300 | 30.0 |
| Barium sulfate | 100 | 10.0 |

The formed article can be used a carrier system for other imaging devices.

### Composition VI

A pharmaceutical composition for imaging applications was prepared according to the method of Example 2, in the form of tablets and other predetermined shapes processed by molding the molten mass into several molds of various shapes and cooling it. The pharmaceutical composition is formed from a fat (Myverol 18-07), a wax (Candelilla wax), a water soluble material (Maltrin 180), and a non-ionic surfactant (Cremophor RH-40). The components and their amounts were as follows:

| Component | Weight (gr) | % (w/w) |
|---|---|---|
| Candelilla wax | 450 | 45.0 |
| Myverol 18-07 | 100 | 10.0 |
| Cremophor RH-40 | 100 | 10.0 |
| Maltrin 180 | 350 | 35.0 |

The formed article can be used a carrier system for other imaging devices.

### Composition VII

A pharmaceutical composition for imaging applications was prepared according to the method of Example 2, in the in the form of particle processed by the cryopelletization procedure that allows conversion of the molten mass into solid, bead-like particles. The pharmaceutical composition is formed from a fat (Glyceryl monostearate), a wax (Carnauba wax) an active ingredient (Barium sulfate), a water soluble materials (Lactitol), and a mixture of a non-ionic surfactant (Tween 20). The components and their amounts were as follows:

| Component | Weight (gr) | % (w/w) |
|---|---|---|
| Carnauba wax | 300 | 30.0 |
| Glyceryl Monostearate | 100 | 10.0 |
| Tween 20 | 150 | 15.0 |
| Lactitol | 350 | 35.0 |
| Barium sulfate | 100 | 10.0 |

### PREPARATION OF CONTROLLED RELEASE COMPOSITIONS

### EXAMPLE 3

Compositions according to the present invention were prepared as follows. The specific components used are detailed in compositions VIII to X. The wax was melted at about ten degrees above the melting point. In a separate vessel the fat was also melted at about ten degrees above the melting point of the wax. The appropriate surfactants are added to the fat melt, while homogenizing the melt at a temperature about ten degrees above the melting point of the wax. The wax and fat melts are combined and the water-soluble materials and the active ingredients are added to the melt slowly while homogenizing the mixture.

### Composition VIII

A controlled release composition was prepared according to the method of Example 3, in the form of particle processed by the cryopelletization procedure that allows conversion of the molten mass into solid, bead-like particles. The pharmaceutical composition is formed from a fat (Glyceryl Monostearate), a wax (Candelilla wax) an encapsulated active ingredient (a flavor, menthol, that has been spray dried with Capsul starch, 50 percent flagrance payload in the spray dried particles), a water soluble material (Maltrin 150), and a non-ionic surfactant (Tween 20). The components and their amounts were as follows:

| Component | Weight (gr) | % (w/w) |
|---|---|---|
| Candelilla wax | 500 | 60.0 |
| Glyceryl monostearate | 100 | 10.0 |
| Tween 20 | 50 | 5.0 |
| Spray-dried menthol particles | 200 | 20.0 |
| Maltrin 150 | 50 | 5.0 |

### Composition IX

A pharmaceutical composition was prepared according to the method of Example 3, in the form of a tablet processed by molding the molten mass into a mold and cooling it. The pharmaceutical composition is formed from a of fat (Glyceryl Monostearate), a wax (Carnauba wax) an encapsulated chemotherapeutic active ingredient in the form of spray dried particles (chemotherapeutic agent is paclitaxel at 10% payload in the particles), a water soluble materials (Lactitol), and a mixture of a non-ionic surfactant (Tween 20). The components and their amounts were as follows:

| Component | Weight (gr) | % (w/w) |
|---|---|---|
| Carnauba wax | 400 | 40.0 |
| Glyceryl monostearate | 50 | 5.0 |
| Tween 20 | 50 | 5.0 |
| Lactitol | 300 | 30.0 |
| Spray-dried paclitaxel particles | 200 | 20.0 |

### Composition X

A pharmaceutical composition was prepared according to the method of Example 3, in the form of a tablet processed by molding the molten mass into a mold and cooling it. The pharmaceutical composition is formed from a of fat (Myverol 18-07), a wax mixture (Carnauba wax and Beeswax) an active (Ibuprofen), a water soluble materials (Lactitol), and a mixture of a non-ionic surfactant (Tween 20). The components and their amounts were as follows:

| Component | Weight (gr) | % (w/w) |
|---|---|---|
| Beeswax wax | 250 | 25.0 |
| Carnauba wax | 200 | 20.0 |
| Myverol 18-07 | 50 | 5.0 |
| Tween 20 | 50 | 5.0 |
| Lactitol | 350 | 35.0 |
| Ibuprofen | 100 | 10.0 |

It is to be understood that the above-described embodiments are illustrative of only a few of the many possible specific embodiments which can represent applications of the principles of the invention. Numerous and varied other arrangements can be readily devised in accordance with these principles by those skilled in the art without departing from the spirit and scope of the invention.

What is claimed is:

1. A composition for controlled release of an active agent comprising:
a wax material;
a fat material;
an active agent;
a water sensitive material; and
optionally a surface active material.

2. The composition of claim 1 wherein said wax material has a melting point in the range of between about 25 degrees C. and about 150 degrees C.

3. The composition of claim 1 wherein said wax material has a penetration point of about 1 to about 10.

4. The composition of claim 1 wherein said wax material is selected from the group consisting of:
natural wax, synthetic wax, regenerated wax, vegetable wax, animal wax, mineral wax, petroleum wax, microcrystalline wax and mixtures thereof.

5. The composition of claim 1 wherein said wax material controls an erosion rate of said composition.

6. The composition of claim 1 wherein said wax comprises one or more of carnauba wax, candelilla wax and beeswax.

7. The composition of claim 1 wherein said fat material is selected from the group consisting of:
hydrogenated castor oil, hydrogenated vegetable oil, hard fat, glyceride, fatty acids, fatty acid derivative, lipid, steroid and mixtures thereof.

8. The composition of claim 7 wherein said glyceride is selected from the group consisting of:
triglyceride, monoglyceride, diglyceride, glyceryl monostearate, glycerol tristearate and mixtures thereof.

9. The composition of claim 7 wherein said fatty acid derivatives are selected from the group consisting of:
alcohol, ester, anhydride, hydroxy fatty acid and prostaglandin.

10. The composition of claim 1 wherein said fat material is selected from the group consisting of:
lauric acid, physeteric acid, myristoleic acid, palmitoleic acid, petroselinic acid, oleic acid, isolauric acid, isomyristic acid, isopalmitic acid, isostearic acid, isoprenoid, 12-(((7′-diethylaminocoumarin-3yl)carbonyl)methylamino)-octadecanoic acid, N-[12-(((7′ diethylaminocoumarin-3-yl)carbonyl)methyl-amino)octadecanoyl]-2-aminopalmitic acid, N succinyl-dioleoylphosphatidylethanol amine, palmitoyl-homocysteine, digalactosyldiglyceride, 1,2-dioleoyl-sn-glycerol; 1,2-cdipalmitoyl-sn-3 succinylglycerol; 1,3-dipalmitoyl-2-succinylglycerol and mixtures thereof.

11. The composition of claim 1 wherein said fat material is selected from the group consisting of:
phospholipid, sphingolipid, cholesterol, steroid derivative, terpene, tocopherol, stearlyamine, vitamin and mixtures thereof.

12. The composition of claim 11 wherein said phospholipid comprises:
phosphatidic acid, phosphatidyl choline, phosphatidyl ethanolamine, phosphatidylglycerol, phosphatidylserine, phosphatidylinositol, lysophosphatidyl derivative, cardiolipin, beta-acyl-y-alkyl phospholipid, phosphatidylcholines, dioleoylphosphatidylcholine, dimyristoylphosphatidylcholine, dipentadecanoylphosphatidylcholine, dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), dilignoceroylphatidylcholine (DLPC), phosphatidylethanolamine, dioleoylphosphatidylethanolamine, 1-hexadecyl-2-palmitoylglycerophosphoethanolamine, synthetic phospholipids and mixtures thereof.

13. The composition of claim 11 wherein said steroid is selected from the group consisting of:
cholesterol, cholesterol sulfate, cholesterol hemisuccinate, 6-(5-cholesterol 3 beta-yloxy) hexyl6-amino-6-deoxy-1-thio-alpha-D-galactopyranoside, 6-(5-cholesten-3 beta-tloxy)hexyl-6-amino-6-deoxyl-1-thio-alpha-D mannopyranoside, cholesteryl(4′-trimethyl 35 ammonio)butanoate and mixtures thereof.

14. The composition of claim 1, wherein said water sensitive material is selected from the group consisting of:
natural oligomer, synthetic oligomer, natural polymer, synthetic polymer and copolymer, starch, starch derivative, oligosaccharide, polysaccharide, hydrocolloid, natural gum, protein, cellulose, cellulose derivative and mixtures thereof.

15. The composition of claim 1, wherein said water sensitive material is selected from the group consisting of:
xylose, ribose, glucose, mannose, galactose, fructose, dextrose, polydextrose, sucrose, maltose, corn syrup, palatin, sorbitol, xylitol, mannitol, maltitol, lactitol, xanthan, maltodextrin, galactomanan, tragacanth and mixtures thereof.

16. The composition of claim 1, wherein said water sensitive material is selected from the group consisting of:
polyvinyl pyrrolidone, water soluble cellulose, polyvinyl alcohol, ethylene maleic anhydride copolymer, methylvinyl ether maleic anhydride copolymer, acrylic acid copolymer, anionic polymer of methacrylic acid and methacrylate, cationic polymer having dimethyl-aminoethyl ammonium functional groups, polyethylene oxide, water soluble polyamide, polyester and mixtures thereof.

17. The composition of claim 1, wherein said water sensitive material is selected from the group consisting of:
hydroxyethyl cellulose, carboxymethyl cellulose, hydroxymethyl cellulose, carboxymethyl cellulose, hydroxypropyl carboxymethyl cellulose, hydroxypropyl methyl carboxyethyl cellulose, hydroxypropyl carboxypropyl cellulose, hydroxybutyl carboxymethyl cellulose and mixtures thereof.

18. The composition of claim 1 wherein said composition comprises said surface active material, said surface active material has an HLB of at least about 10.

19. The composition of claim 1 wherein said composition includes said surface active material, said surface active material has an HLB value in a range of about 15 to about 50.

20. The composition of claim 1 wherein said composition comprises said surface active material, said surface active material is selected from the group consisting of:
an anionic surfactant, a cationic surfactant, a nonionic surfactant, an amphoteric surfactant and mixtures thereof.

21. The composition of claim 20 wherein said anionic surfactant comprises a carboxylate, sulfonate and sulfate ion.

22. The composition of claim 20 wherein said anionic surfactant is selected from the group consisting of:
alkyl carboxylate, acyl lactylate, alkyl ether carboxylate, N-acyl sarcosinate, polyvalent alkyl carbonate, N-acyl glutamate, fatty acid, polypeptide condensate, sulfuric acid ester and mixtures thereof.

23. The composition of claim 20 wherein said nonionic surfactant is selected from the group consisting of:
polyoxyethylene, lecithin, ethoxylated alcohol, ethoxylated ester, ethoxylated amide, polyoxypropylene, propoxylated alcohol, ethoxylated/propoxylated block polymer, propoxylated ester, alkanolamide, amine oxide, fatty acid esters of polyhydric alcohol, ethylene glycol ester, diethylene glycol ester, propylene glycol ester, glycerol ester, polyglycerol fatty acid ester, sorbitan ester, sucrose ester, glucose ester, dextrose ester, simethicone and mixtures thereof.

24. The composition of claim 1 wherein said composition comprises said surface active material, said surface active material is selected from the group consisting of:
acacia, benzalkonium chloride, cholesterol, emulsifying wax, glycerol monostearate, lanolin alcohols, lecithin, poloxamer, polyoxyethylene, castor oil derivatives and mixtures thereof.

25. The composition of claim 1 wherein said composition comprises said surface active material, said surface active material is selected from the group consisting of:
calcium sulfate, barium sulfate and sodium lauryl sulfate.

26. The composition of claim 1 further comprising a pH sensitive material, wherein said pH sensitive material releases said composition only upon trigger by an environment having a trigger pH.

27. The composition of claim 26 wherein said trigger pH is in the range of about 3 to about 12.

28. The composition of claim 26 wherein said pH sensitive material is selected from the group consisting of:
acrylate polymers with amino substituents, acrylic acid esters, polyacrylamides, phthalate derivatives and mixtures thereof.

29. The composition of claim 26 wherein said pH sensitive material is selected from the group consisting of:
acid phthalate of carbohydrate, amylose acetate phthalate, cellulose acetate phthalate, cellulose ester phthalate, cellulose ether phthalate, hydroxy propyl cellulose phthalate, hydroxypropyl ethylcellulose phthalate, hydroxypropyl methyl cellulose phthalate, methyl cellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinyl acetate phthalate copolymer, styrene and maleic acid copolymer, gelatin, gluten, shellac, salol, keratin, keratin sandarac-tolu, ammoniated shellac, benzophenyl salicylate, cellulose acetate trimellitate, cellulose acetate blended with shellac, hydroxypropylmethyl cellulose acetate succinate, oxidized cellulose, polyacrylic acid derivative, acrylic acid and acrylic ester copolymers, methacrylic acid, methacrylic acid ester, vinyl acetate, crotonic acid copolymer and mixtures thereof.

30. The composition of claim 26 wherein said pH sensitive material is relatively insoluble and impermeable at the pH of the stomach and is more soluble and permeable at the pH of the small intestine and colon.

31. The composition of claim 1 wherein said active agent is selected from the group consisting of:
analgesic, antihistamine, anti-emetic, anti-epileptic, vasodilator, anti-tussive agent, expectorant, anti-hypotensive, anti-inflammatory agent, anthelmintic, anti-arrhythmic agent, anti-bacterial agent, anti-viral agent, anti-coagulant, anti-depressant, anti-diabetic, anti-epileptic, anti-fungal agent, anti-gout agent, anti-hypertensive agent, anti-malarial, anti-migraine agent, anti-muscarinic agent, anti-neoplastic agent, anti-stroke agent, erectile dysfunction improvement agent, immunosuppressant, anti-protozoal agent, anti-thyroid agent, anxiolytic agent, sedative, hypnotic, neuroleptic, beta-Blocker, cardiac inotropic agent, corticosteroid, diuretic, anti-parkinsonian agent, gastro-intestinal agent, histamine receptor antagonist, keratolytic, lipid regulating agent, anti-anginal agent, cox-2-inhibitor, leukotriene inhibitor, macrolide, muscle relaxant, nutritional agent, opioid analgesic, protease inhibitor, sex hormone, muscle relaxant, anti-osteoporosis agent, anti-obesity agent, cognition enhancer, anti-urinary incontinence agent, nutritional oil, anti-benign prostate hypertrophy agent, essential fatty acid, non-essential fatty acid, antihemorrhoidal, psychotropic, antidiarrheal, mucolytic, decongestant, laxative, vitamin, stimulant, appetite suppressant, contraceptive, protein, peptide, sugar, natural agent and mixtures thereof.

32. The composition of claim 1 wherein said active agent is one or more of a pharmaceutical agent, cosmetic substance, drug or steroid.

33. The composition of claim 1 wherein said active agent is selected from the group consisting of:
antibiotic, antiviral, antigen, vaccine, vasodilator, vasoconstrictor, immunomodulatory compound, cytokine, colony stimulating factor, tumor necrosis factor, interferon, oligonucleotide, nuclease, bronchodilator, hormone, calcitonin, insulin, erthropoietin, growth hormone and mixtures thereof.

34. The composition of claim 1 wherein said active agent is delivered to the colon.

35. The composition of claim 34 wherein said active agent is selected from the group consisting of:
nonsteroidal anti-inflammatory drug (NSAID), and aspirin; steroid, contraceptive, steroidal hormone, immunosuppressant, bronchodialators, anti-anginal, anti-hypertensive anti-spasmodic agent, anti-colitis agent, anti-arrhythmia, anti-neoplastic agent, protein, peptide drug, interferon, calcitonin, leuprolide, tumor necrosis factor, bone growth factor, melanocyte-stimulating hormone, captopril, somatostatin, somatostatin octapeptide analog, cyclosporin, renin inhibitor, superoxide dismutase, vaccine, anticoagulant, anti-migraine drug, antagonist ondasteron, menthol, antibiotic, beta-lactam, cephalosporin, macrolide, analogues for protecting the gastroduodenal mucosa from NSAID injury and mixtures thereof.

36. The composition of claim 1 wherein said active agent is delivered to an alimentary canal.

37. The composition of claim 1 wherein said active agent is delivered to the stomach and/or small intestine.

38. The composition of claim 1 wherein said active agent is delivered to the gastrointestinal tract.

39. The composition of claim 1 wherein said active agent is an imaging agent.

40. The composition of claim 39 wherein said imaging agent is one or more of barium sulfate, siatrizoate sodium iodine containing contrast agents, ultra sound contrast agents, magnetic resonance imaging contrast agents, magnetic resonance imaging enhancements, tomography agents, and positron emission agents.

41. The composition of claim 1 wherein said active agent is a monoclonal antibody marker.

42. The composition of claim 1 wherein the matrix compositions of the present invention can comprises from about 1% to about 50% by weight of wax, from about 1% to about 50% of fat, from about 1% to about 50% by weight water sensitive matrix, from about 1% to about 50% by weight active ingredients, and from about 0% to about 30% by weight surface active agents.

43. The composition of claim 1 wherein said composition dissolves or swells upon contact with moisture.

44. The composition of claim 1 wherein said active agent is encapsulated in a water sensitive microsphere.

45. The composition of claim 44 wherein said water sensitive microsphere is formed of a material selected from the group consisting of: natural oligomer, synthetic oligomer, natural polymer, synthetic polymer and copolymer, starch, starch derivative, oligosaccharide, polysaccharide, hydrocolloid, natural gum, protein, cellulose, cellulose derivative, xylose, ribose, glucose, mannose, galactose, fructose, dextrose, polydextrose, sucrose, maltose, corn syrup, palatin, sorbitol, xylitol, mannitol, maltitol, lactitol, xanthan, maltodextrin, galactomanan or tragacanth, polyvinyl pyrrolidone, water soluble cellulose, polyvinyl alcohol, ethylene maleic anhydride copolymer, methylvinyl ether maleic anhydride copolymer, acrylic acid copolymer, anionic polymer of methacrylic acid and methacrylate, cationic polymer having dimethyl-aminoethyl ammonium functional groups, polyethylene oxide, water soluble polyamide, polyester, hydroxyethyl cellulose, carboxymethyl cellulose, hydroxymethyl cellulose, carboxymethyl cellulose, hydroxypropyl carboxymethyl cellulose, hydroxypropyl methyl carboxyethyl cellulose, hydroxypropyl carboxypropyl cellulose, hydroxybutyl carboxymethyl cellulose and mixtures thereof.

46. The composition of claim 44 wherein said water sensitive material comprises said water sensitive microsphere.

47. The composition of claim 44 wherein the water sensitive microsphere further comprises a bioadhesive material.

48. The composition of claim 44 wherein said microsphere has a size from about 2 microns to about 30 microns.

49. The composition of claim 1 wherein said active agent is encapsulated in a multicomponent carrier comprising solid hydrophobic nanospheres encapsulated in moisture sensitive or pH sensitive microspheres.

50. The composition of claim 49 wherein said active ingredients are encapsulated in said hydrophobic nano-spheres.

51. The composition of claim 49 wherein said active ingredients are encapsulated in said hydrophobic nanospheres, said moisture sensitive microspheres or pH sensitive microspheres.

52. The composition of claim 49 wherein said active ingredients are encapsulated in said hydrophobic nano-spheres, both hydrophobic nanospheres and said moisture sensitive microspheres or pH sensitive micropheres.

53. The composition of claim 49 wherein said nanospheres comprise a cationic surface active agent, anionic surface active agent, a nonionic surface active agent or a zwitterionic surface active agent.

54. The composition of claim 49 wherein said nanoparticles further comprise a bioadhesive material.

55. An article formed of the composition of claim 1.

56. The article of claim 55 wherein said article is selected from an implant, micro-implant, micro-particle, micro-sphere, micro-capsule, sheet, film, tube and medical device.

57. The article of claim 55 wherein said article is a dental bone implant.

58. The article of claim 55 wherein said article is formed by granulation, melt fabrication, extrusion, spray congealing, cryopelletization or spheronation of said composition.

59. The article of claim 55 wherein said active agent is an imaging agent that is administrable in the stomach and/or small intestine.

60. The article of claim 55 wherein said active agent is a drug that is administrable in the stomach and/or small intestine.

61. The article of claim 55 wherein said composition further comprises a device, said article being a carrier for said device.

62. The article of claim 55 wherein said device is an implant device, imaging device or medical device.

63. The article of claim 55 wherein the shape and surface area of said article controls a release rate of said active agent.

64. A method for delivering an active substance to a preselected environment; said method comprising introducing to said environment a control release composition, said control release comprising:
a wax material;
a fat material;
an active agent;
a water sensitive material; and
optionally a surface active material,
wherein introducing of said composition into said environment permits degradation of said composition and release of said active agent.

65. The method of claim 64 wherein said environment is the stomach or small intestine.

66. The method of claim 64 wherein said wax material is selected from the group consisting of:
natural wax, synthetic wax, regenerated wax, vegetable wax, animal wax, mineral wax, petroleum wax, microcrystalline wax and mixtures thereof.

67. The method of claim 64 wherein said fat material is selected from the group consisting of:
hydrogenated castor oil, hydrogenated vegetable oil, hard fat, glyceride, fatty acids, fatty acid derivative, lipid, steroid, phospholipid, sphingolipid, cholesterol, steroid derivative, terpene, tocopherol, stearlyamine, vitamin and mixtures thereof.

68. The method of claim 64 comprising said water sensitive material, wherein said water sensitive material is selected from the group consisting of:
natural oligomer, synthetic oligomer, natural polymer, synthetic polymer and copolymer, starch, starch derivative, oligosaccharide, polysaccharide, polyvinyl pyrrolidone, polyvinyl alcohol, hydrocolloid, natural gum, protein, cellulose, cellulose derivative and mixtures thereof.

69. The method of claim 64 wherein said composition further comprises said surface active material, said surface active material is selected from the group consisting of:
an anionic surfactant, a cationic surfactant, a nonionic surfactant, an amphoteric surfactant and mixtures thereof.

70. The method of claim 64 wherein said active agent is selected from the group consisting of:
analgesic, antihistamine, anti-emetic, anti-epileptic, vasodilator, anti-tussive agent, expectorant, anti-hypotensive, anti-inflammatory agent, anthelmintic, anti-arrhythmic agent, anti-bacterial agent, anti-viral agent, anti-coagulant, anti-depressant, anti-diabetic, anti-epileptic, anti-fungal agent, anti-gout agent, anti-hypertensive agent, anti-malarial, anti-migraine agent, anti-muscarinic agent, anti-neoplastic agent, anti-stroke agent, erectile dysfunction improvement agent, immunosuppressant, anti-protozoal agent, anti-thyroid agent, anxiolytic agent, sedative, hypnotic, neuroleptic, beta-Blocker, cardiac inotropic agent, corticosteroid, diuretic, anti-parkinsonian agent, gastro-intestinal agent, histamine receptor antagonist, keratolytic, lipid regulating agent, anti-anginal agent, cox-2-inhibitor, leukotriene inhibitor, macrolide, muscle relaxant, nutritional agent, opioid analgesic, protease inhibitor, sex hormone, muscle relaxant, anti-osteoporosis agent, anti-obesity agent, cognition enhancer, anti-urinary incontinence agent, nutritional oil, anti-benign prostate hypertrophy agent, essential fatty acid, non-essential fatty acid, antihemorrhoidal, psychotropic, antidiarrheal, mucolytic, decongestant, laxative, vitamin, stimulant, appetite suppressant, contraceptive, protein, peptide, sugar, natural agent and mixtures thereof.

71. The method of claim 64 wherein said active agent is one or more of a pharmaceutical agent, cosmetic substance, drug or steroid.

72. The method of claim 64 wherein said active agent is selected from the group consisting of:
antibiotic, antiviral, antigen, vaccine, vasodilator, vasoconstrictor, immunomodulatory compound, cytokine, colony stimulating factor, tumor necrosis factor, interferon, oligonucleotide, nuclease, bronchodilator, hormone, calcitonin, insulin, erthropoietin, growth hormone and mixtures thereof.

73. The method of claim 64 wherein said active agent is delivered to the colon.

74. The method of claim 64 wherein said active agent is delivered to an alimentary canal.

75. The method of claim 64 wherein said active agent is delivered to the stomach and/or small intestine.

76. The method of claim 64 wherein said active agent is delivered to the gastrointestinal tract.

77. The method of claim 64 wherein said active agent is an imaging agent.

78. The method of claim 64 wherein said imaging agent is one or more of barium sulfate, siatrizoate sodium and iodine containing contrast agents.

79. The composition of claim 1 wherein said active agent is encapsulated in a water sensitive microsphere.

80. The composition of claim 79 wherein said water sensitive microsphere is formed of a material selected from the group consisting of: natural oligomer, synthetic oligomer, natural polymer, synthetic polymer and copolymer, starch, starch derivative, oligosaccharide, polysaccharide, hydrocolloid, natural gum, protein, cellulose, cellulose derivative, xylose, ribose, glucose, mannose, galactose, fructose, dextrose, polydextrose, sucrose, maltose, corn syrup, palatin, sorbitol, xylitol, mannitol, maltitol, lactitol, xanthan, maltodextrin, galactomanan, tragacanth, polyvinyl pyrrolidone, water soluble cellulose, polyvinyl alcohol, ethylene maleic anhydride copolymer, methylvinyl ether maleic anhydride copolymer, acrylic acid copolymer, anionic polymer of methacrylic acid and methacrylate, cationic polymer having dimethyl-aminoethyl ammonium functional groups, polyethylene oxide, water soluble polyamide, polyester, hydroxyethyl cellulose, carboxymethyl cellulose, hydroxymethyl cellulose, carboxymethyl cellulose, hydroxypropyl carboxymethyl cellulose, hydroxypropyl methyl carboxyethyl cellulose, hydroxypropyl carboxypropyl cellulose, hydroxybutyl carboxymethyl cellulose and mixtures thereof.

81. The composition of claim 79 wherein the water sensitive microsphere further comprises a bioadhesive material.

82. The composition of claim 79 wherein said microsphere has a size from about 2 microns to about 30 microns.

83. The composition of claim 1 wherein said active agent is encapsulated in a multicomponent carrier comprising solid hydrophobic nanospheres encapsulated in moisture sensitive or pH sensitive microspheres.

84. The composition of claim 83 wherein said active ingredients are encapsulated in said hydrophobic nano-spheres.

85. The composition of claim 83 wherein said active ingredients are encapsulated in said hydrophobic nanospheres, said moisture sensitive microspheres or pH sensitive microspheres.

86. The composition of claim 83 wherein said active ingredients are encapsulated in said hydrophobic nano-spheres, both hydrophobic nanospheres and said moisture sensitive micro spheres or pH sensitive micropheres.

87. The composition of claim 83 wherein said nanospheres comprise a cationic surface active agent, anionic surface active agent, a nonionic surface active agent or a zwitterionic surface active agent.

88. The composition of claim 83 wherein said nanoparticles further comprise a bioadhesive material.

89. A method for producing a controlled release composition comprising the steps of:
(i) heating a wax material to a temperature above the melting point of the wax material to form a melt;
(ii) heating a fat material to a temperature above the melting point of the fat material to form a melt;
(iii) dissolving or dispersing a surface active agent into the melt of the wax or melt of the fat;
(iv) combining the melt of the wax with the melt of the fat;
(v) dissolving or dispersing one or more first active agents into the combined melt of the wax and fat;
(vi) optionally dissolving or dispersing one or more second encapsulated active agents into the combined melt of the wax and fat;
(vii) dispersing a water sensitive material into the combined melt of the wax and fat; and
(viii) cooling the melt to form a dry powder composition.

90. A method for producing articles formed of a controlled release composition comprising the steps of:
(i) heating a wax material to a temperature above the melting point of the wax material to form a melt;
(ii) heating a fat material to a temperature above the melting point of the fat material to form a melt;
(iii) dissolving or dispersing a surface active agent into the melt of the wax or melt of the fat;
(iv) combining the melt of the wax with the melt of the fat;
(v) dissolving or dispersing one or more first active agents into the combined melt of the wax and fat;
(vi) optionally dissolving or dispersing one or more second encapsulated active agents into the combined melt of the wax and fat;
(vii) dispersing a water sensitive material into the combined melt of the wax and fat;
(viii) cooling the melt to form a dry powder composition; or
(ix) molding the melt into a predetermined geometric dimensions or shapes.

91. A composition for controlled release of a device comprising:
a wax material;
a fat material;
an active agent;
a water sensitive material; and
said device, wherein said composition is a carrier for said device.

92. The composition of claim 91 wherein said device is an implant device, imaging device or a medical device.

93. An article of manufacture formed of the composition of claim 90.

94. The composition of claim 93 wherein said device is an implant device, imaging device or a medical device.
